(19) United States
(12) Patent Application Publication
(10) Pub. No.: US 2011/0003994 A1
(43) Pub. Date: Jan. 6, 2011

(54) CYCLOALKANE DERIVATIVE

(75) Inventor: Megumi MARUYAMA, Osaka-shi (JP)

Correspondence Address: BIRCH STEWART KOLASCH & BIRCH, PO BOX 747, FALLS CHURCH, VA, 22040-0747, US

(73) Assignee: Dainippon Sumitomo Pharma Co., Ltd.

(21) Appl. No.: 12/828,130

(22) Filed: Jun. 30, 2010

Related U.S. Application Data
(60) Provisional application No. 61/222,775, filed on Jul. 2, 2009.

(51) Int. Cl. C07D 417/14; C07D 413/04; C07D 401/04; C07D 401/14; C07D 403/08

(52) U.S. Cl. 544/368; 546/198; 546/199; 546/201; 544/372

(57) ABSTRACT

The present invention relates to a novel cycloalkane derivative which exhibits an excellent psychotropic action and has less side effect.

### TECHNICAL FIELD

The present invention relates to a novel cycloalkane derivative and an acid addition salt thereof which are useful as a psychotropic compound. In more detail, the compound of the present invention is useful as a medicament for treating, for example, schizophrenia, senile insanity, bipolar disorder, depression, neurosis, senile dementia and associated symptoms thereof.

### BACKGROUND ART

Patent References 1-3 disclose some cycloalkane derivatives which have psychotropic action.

The compounds disclosed in Patent References 1 and 2 are different from the derivatives of the present invention on the structures of group D and aromatic heterocyclyl group Ar attached to the piperazine ring in the general formula [1] of the present invention.

In addition, the compounds disclosed in Patent Reference 3, which have a cyclic amide structure in the side chain, are different from the derivatives of the present invention.

Furthermore, psychotropic drugs which have been currently used can be accompanied with some disorders such as side effects in CNS, extrapyramidal disorder (e.g. catalepsy), oversedation, as well as cognitive decline. Consequently, the disorders of such drugs have been a serious problem in clinical field (Non-patent Reference 1).

### PRIOR ART

### Patent Reference

[Patent Reference 1] JP-5 (1993)-17440 A

[Patent Reference 2] JP-8 (1996)-333368 A

[Patent Reference 3] WO 96/14297

### [Non-Patent Reference]

[Non-patent Reference 1] The Pharmacological basis of therapeutics, A. Goodman Gilman, L. S. Goodman et al, New York (1985) P 387, GENDAI IRYO, 22, P 22, (1990)

### DISCLOSURE OF INVENTION

### Problem to be Solved by the Invention

The purpose of the present invention is to provide a good psychotropic drug which has less side effect. Especially, the purpose is to provide a psychotropic drug which exhibits an excellent effect for improving a broad spectrum of schizophrenia such as positive symptom, negative symptom, and cognitive dysfunction, while never almost causing abnormal electrocardiogram, weight gain, increased blood glucose, etc., i.e. the desired drug is very safe and could be administered for a long term.

### Means to Solve the Problem

The present inventor has extensively studied to reach the above object and then has found that the novel cycloalkane derivatives of the present invention exhibit the desired pharmacological actions and further reduce the side effects. Based upon the new findings, the present invention has been completed.

The present invention relates to the following inventions.

### Term 1

A cycloalkane derivative of formula [1]

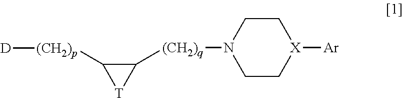

wherein

p and g are independently 1 or 2;

T is —(CH₂)ₙ— wherein n is 1 to 4, or —C(═CH₂)—;

D is the group of formula [2]:

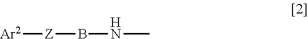

wherein Ar² is an aromatic heterocyclyl group or an aromatic carbocyclyl group wherein the aromatic heterocyclyl group and the aromatic carbocyclyl group may be substituted with 1 or 2 substituents selected independently from the group consisting of nitro group, cyano group, halogen atom, lower alkyl group, lower alkoxy group, trifluoromethyl group, trifluoromethoxy group and phenoxy group, and further the lower alkyl group, the alkoxy group and the phenoxy group may be substituted with one or more halogen atoms which are the same or different;

B is carbonyl group or sulfonyl group,

Z is single bond, lower alkylene, lower alkenylene, or ethynylene,

the group of formula [3]:

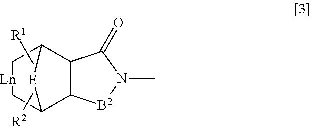

wherein B² is carbonyl group or sulfonyl group;

Ln is single or double bond;

E is lower alkylene which may be optionally substituted with one or two lower alkyl groups which are the same or different, oxygen atom, or two hydrogen atoms which are attached at the both ends (i.e., E is not a bridge);

R¹ and R² are independently hydrogen atom, hydroxy, lower alkyl group, or lower cycloalkyl group, which may be independently connected to any one of the carbon atoms which compose the ring of formula [3], or R¹ and R² may be connected to the same carbon atom if possible, wherein the lower alkyl group and the lower cycloalkyl group may be substituted with one or more substituents selected independently from the group consisting of hydroxy group and fluorine atom, or

the group of formula [4]:

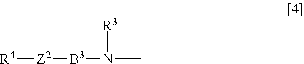

wherein B³ is carbonyl group or sulfonyl group;

Z² is single bond, oxygen atom, or —NR⁵—;

R³, R⁴ and R⁵ are independently hydrogen atom or lower alkyl, or R³ and R⁴, or R⁴ and R⁵ may be connected together directly or via lower alkylene to form a ring;

X is N, CH or C(OH); and

Ar is aromatic heterocyclyl group, aromatic hydrocarbon group, benzoyl, or phenoxy, wherein the aromatic heterocyclyl group, the aromatic hydrocarbon group, the benzoyl, and the phenoxy may be substituted with one or more substituents selected independently from the group consisting of lower alkyl, lower alkoxy and halogen atom;

provided that Ar is not benzisothiazolyl group when D is the substituent of formula [3],

or an acid additive salt thereof.

### Term 2

The cycloalkane derivative of Term 1

wherein

p and q are 1,

T is —(CH₂)ₙ— wherein n is 1 to 4,

D is the group of formula [2]:

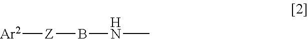

wherein Ar² and B are defined as Term 1, and Z is single bond, methylene, vinylene or ethynylene, or

the group of formula [3]:

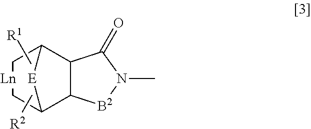

wherein B², E, R¹, R² and Ln are defined as Term 1, and

X is N or CH,

or an acid additive salt thereof.

### Term 3

The cycloalkane derivative of Term 2

wherein

T is —(CH₂)ₙ— wherein n is 3 or 4,

D is the group of formula [2]:

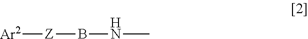

wherein Ar² and B are defined as Term 2, and Z is single bond, methylene or vinylene,
or an acid additive salt thereof.

### Term 4

The cycloalkane derivative of Term 2 or 3 wherein Z is single bond or vinylene, or an acid additive salt thereof.

### Term 5

An antipsychotic agent comprising the cycloalkane derivative of any one of Terms 1-4 or an acid addition salt thereof.

### Term 6

A method for treating psychosis comprising administering an effective amount of the cycloalkane derivative of any one of Terms 1-4 or an acid addition salt thereof to a mammal in need thereof.

### Term 7

Use of the cycloalkane derivative of any one of Terms 1-4 or an acid addition salt thereof in preparation of an antipsychotic agent.

### BEST MODE FOR CARRYING OUT THE INVENTION

Hereinafter, the functional groups used herein are illustrated.

The lower alkylene group used in Z and E, or used as a bridge in case that R³ and R⁴, or R⁴ and R⁵ are connected together to form a ring, is, for example, C₁₋₆ alkylene group, and preferably C₁₋₃ alkylene group, including methylene, ethylene, trimethylene, etc. The more preferable alkylene is methylene.

The lower alkenylene group used in Z is, for example, C₂₋₆ alkenylene group, and preferably C₂₋₃ alkenylene group, including vinylene, propenylene, etc. The more preferable alkenylene is vinylene.

The aromatic hydrocarbon group used in Ar and Ar² is, for example, aromatic hydrocarbon group having 6 to 14 carbon atoms, and preferably having 6 to 10 carbon atoms, including phenyl, naphthyl, etc. The more preferable aromatic hydrocarbon group is phenyl.

The aromatic heterocyclyl group used in Ar and Ar² includes, for example, monocyclic aromatic heterocyclyl group, and bicyclic aromatic heterocyclyl group.

The monocyclic aromatic heterocyclyl group includes, for example, an aromatic ring group having not more than 6 carbon atoms and including 1 to 4 heteroatoms selected independently from nitrogen atom, oxygen atom or sulfur atom, and for example, pyridyl, pyrimidinyl, thiazolyl, oxazolyl, isoxazolyl, isothiazolyl, furyl, imidazolyl, etc. are exemplified.

The bicyclic aromatic heterocyclyl group includes, for example, a bicyclic group having not more than 10 carbon atoms and including 1 to 5 heteroatoms selected independently from nitrogen atom, oxygen atom or sulfur atom, wherein the heteroatoms may be contained in only one ring, in both rings, or in lapped site of both rings. The examples thereof include a fused benzologue ring such as benzisoxazolyl, benzofuryl, quinolyl, isoquinolyl, indolyl, indazolyl, indolinyl, oxoindolinyl, tetrahydroquinolyl, dihydroquinolonyl, tetrahydroquinolonyl, benzimidazolyl, and benzoxazolyl; azaindolyl; naphthyridinyl; pteridinyl; thienofuranyl; imidazothiophenyl; imidazofuranyl; benzisothiazolyl; etc. The preferable Ar includes benzoyl, benzisoxazolyl, indazolyl, indolyl, indolinyl, and benzisothiazolyl.

The lower alkyl group used herein is, for example, C₁₋₆ alkyl group, and preferably C₁₋₄ alkyl group, including methyl, ethyl, propyl, 2-propyl, butyl, etc. The more preferable alkyl is methyl and ethyl.

The lower cycloalkyl group used herein is, for example, C₃₋₆ cycloalkyl group, and preferably C₃₋₄ cycloalkyl group, including cyclopropyl, cyclobutyl, etc. The more preferable cycloalkyl is cyclopropyl.

The lower alkyl group substituted with one or more hydroxy groups include, for example, hydroxymethyl group, 2-hydroxyethyl group, 2-hydroxy-2-propyl group, etc.

The lower cycloalkyl group substituted with one or more hydroxy groups include, for example, 2-hydroxycyclo-propyl group, 3-hydroxycyclobutyl group, etc.

The lower alkyl group substituted with one or more fluorine atoms include, for example, trifluoromethyl group, 2-fluoroethyl group, etc.

The lower cycloalkyl group substituted with one or more fluorine atoms include, for example, 2-fluorocyclo-propyl group, 3-fluorocyclobutyl group, etc.

The lower alkoxy group used herein is, for example, alkoxy group, and preferably C₁₋₄ alkoxy group, including methoxy, ethoxy, propoxy, 2-propoxy, butoxy, methylenedioxy, etc. The more preferable lower alkoxy group is methoxy.

The halogen atom used herein includes, for example, fluorine, chlorine, bromine, and iodine. The preferable halogen is fluorine and chlorine.

The group according to formula [4]:

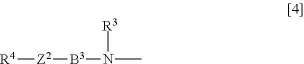

includes, for example, the following groups:

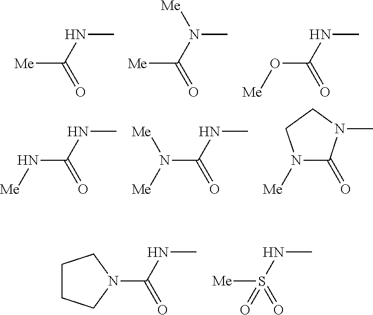

R¹ and R² which are the substituents in formula [3] each may be connected to any carbon atoms of which the ring in formula [3] is composed. R¹ and R² may be connected to the same carbon atom, if possible. In case that E is a lower alkylene, R¹ and R² each may be connected to the carbon atoms of which the lower alkylene is composed.

The acid additive salt used herein includes an addition salt with a pharmaceutically acceptable inorganic acid or organic acid. The salt with an inorganic acid includes, for example, hydrochloride, hydrobromide, hydroiodide, sulfate, nitrate, phosphate, etc. and preferably hydrochloride. The salt with an organic acid includes, for example, acetate, oxalate, citrate, malate, tartrate, maleate, fumarate, etc.

The compound of formula [1] includes any possible stereoisomer(s) and/or optical isomer(s). The compound of the present invention may include a mixture of the isomers and an isolated isomer thereof.

The present compounds [1] can be prepared, for example, by the method shown in the following schemes.

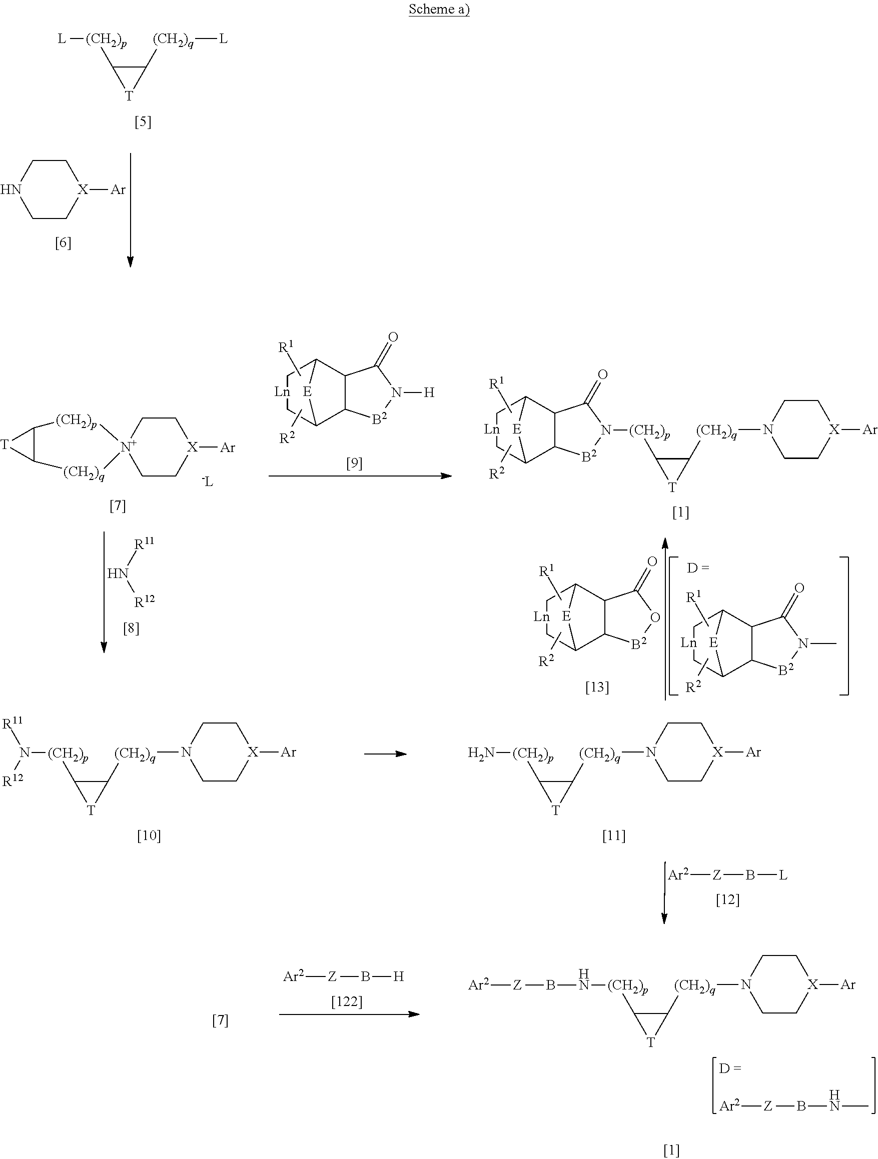

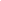

Wherein p, q, R¹, R², D, T, X, Z, B, B², E, Ln, Ar and Ar² are as defined above. R¹¹ and R¹² mean a substituent or a functional group which independently or collectively can act as a protective group and be removed by means of a general chemical procedure to transform the protected amino group to the corresponding free amino group. The compound of formula [8] includes, for example, dibenzylamine, diallylamine, phthalimide, etc.

L denotes a leaving group. The leaving group used herein includes, for example, a halogen atom, an alkylsulfonyloxy, and an arylsulfonyloxy. The alkylsulfonyloxy includes, for example, methanesulfonyloxy, etc. The arylsulfonyloxy includes, for example, p-toluenesulfonyloxy, benzenesulfonyloxy, etc.

The starting compound in Scheme a) is a known compound or can be prepared by the following methods described in some references. For example, the process of the compound of formula [6] is disclosed in JP-63 (1988)-83085 A, J. Med. Chem., 28, 761-769, (1985). And the process of the compound of formula [5] is disclosed in JP-5 (1993)-17440 A.

In case that D denotes the substituent of formula [3];

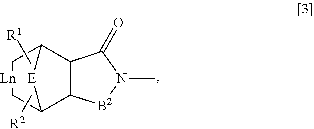

the compound of formula [1] can be prepared by reacting the compound of formula [7] and the compound of formula [9] in the presence of a base and an optional catalyst. The solvent used in the reaction includes, for example, an aromatic solvent such as toluene, xylene, and chlorobenzene. The reaction temperature may be around the boiling point of the reaction solvent. The catalyst used in the reaction includes, for example, a crown ether such as dibenzo-18-crown-6-ether. The amount of the catalyst may be used in the range of 0.1 to 10% per the compound of formula [7] by weight. The amount of the compound of formula [9] may be used in the range of 1 to 1.5 moles per 1 mole of the compound of formula [7].

The compound of formula [7] can be prepared by reacting the compound of formula [5] and the compound of formula [6] in the presence of a base. The solvent used in the reaction includes, for example, an alcohol, acetonitrile, dimethylformamide, etc. The reaction temperature may be around the boiling point of the reaction solvent. The base used in the reaction includes, for example, potassium carbonate, sodium carbonate, etc. The amount of the base may be used in the range of 0.5 to 2 moles per 1 mole of the compound of formula [5]. The amount of the compound of [6] may be used in the range of 1 to 1.5 moles per 1 mole of the compound of formula [5]. The alcohol mentioned above includes, for example, methanol, ethanol, propanol, 2-propanol, butanol, etc.

In addition, the compound of formula [1] can be also prepared by reacting the compound of formula [11] and the compound of formula [13]. The solvent used in the reaction includes, for example, pyridine, toluene, xylene, chlorobenzene, etc. The reaction temperature may be around the boiling point of the reaction solvent.

The compound of formula [11] can be obtained by the deprotection of the compound of formula [10] through a conventional method, for example, the method described in “Protective group in Organic Synthesis, Theodora W. Greene, John Wiley & Sons”.

The compound of formula [10] can be prepared by reacting the compound of formula [7] and the compound of formula [8] optionally in the presence of a base and/or a catalyst. It is possible to use an alkaline metal salt or alkaline earth metal salt of the compound of formula [8], instead of the combination of the compound of formula [8] and a base. The solvent used in the reaction includes, for example, an aromatic solvent such as toluene, xylene, and chlorobenzene; and an aprotic polar solvent such as dimethylsulfoxide and dimethylformamide. The reaction temperature may be around the boiling point of the reaction solvent. The catalyst used in the reaction includes, for example, a crown ether such as dibenzo-18-crown-6-ether. The amount of the catalyst may be used in the range of 0.1 to 10% per the compound of formula [7] by weight. The amount of the compound of formula [8] may be used in the range of 1 to 1.5 moles per 1 mole of the compound of formula [7]. The base used in the reaction includes, for example, an inorganic base such as potassium carbonate and sodium carbonate.

In case that D denotes the substituent of formula [2]:

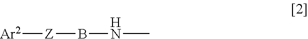

the compound of formula [1] can be prepared by reacting the compound of formula [11] and the compound of formula [12] in the presence of a base and an optional catalyst. The solvent used in the reaction includes, for example, an aprotic polar solvent such as dimethylsulfoxide and dimethylformamide; and a halogen solvent such as chloroform and dichloromethane. The reaction temperature may be around the boiling point of the reaction solvent. The catalyst used in the reaction includes, for example, N,N-dimethylaminopyridine. The amount of the catalyst may be used in the range of 0.1 to 10% per the compound of formula [11] by weight. The amount of the compound of formula [12] may be used in the range of 1 to 1.5 moles per 1 mole of the compound of formula [11]. The base used in the reaction includes, for example, an inorganic base such as potassium carbonate and sodium carbonate; and a tert-amine such as triethylamine and Hunig's base.

In addition, the compound of formula [1] can be prepared by reacting the compound of formula [7] and the compound of formula [122] in the presence of a base and an optional catalyst. The solvent used in the reaction includes, for example, an aromatic solvent such as toluene, xylene, and chlorobenzene. The reaction temperature may be around the boiling point of the reaction solvent. The catalyst used in the reaction includes, for example, a crown ether such as dibenzo-18-crown-6-ether. The amount of the catalyst may be used in the range of 0.1 to 10% per the compound of formula [7] by weight. The amount of the compound of formula [122] may be used in the range of 1 to 1.5 moles per 1 mole of the compound of formula [7].

In case that R¹, R², Ar and Ar² are unstable at each reaction step in the preparation process, these functional groups may be protected through a conventional method, for example, the method described in “Protective group in Organic Synthesis, Theodora W. Greene, John Wiley & Sons”, and each protective group can be cleaved after each reaction is completed.

The protective group for hydroxy group includes, for example, benzyl group, tetrahydropyranyl group, acetyl group, etc. The protective group for amino group includes, for example, benzyl group, tert-butoxycarbonyl group, trifluoroacetyl group, etc. The group that can be easily transformed to amino group, for example, nitro group or the like can be used instead of a protected amino group.

The protective group for carboxyl group, or the functional group that can be easily transformed to carboxyl group includes tert-butyl group, trialkylsilyl group, alkoxycarbonyl group, carbamoyl group, nitrile group, etc.

In case that the compound of formula [1] is optically divided, it can be done as follows. The compound of formula [1] is dissolved in an inert solvent (e.g. acetonitrile, an alcohol, etc.), and then an optically active acid (e.g. L-tartaric acid, D-tartaric acid, D-camphoric acid, L-mandelic acid, L-pyroglutamic acid, D-10-camphorsulfonic acid, D-quinic acid, L-malic acid, dibenzoyl-L-tartaric acid, etc., and preferably L-tartaric acid or D-tartaric acid) is added to the solution to form a salt thereof. The temperature to form the salt can be chosen from the range of room temperature to boiling point of the used solvent. It is preferable to heat the solution around the boiling point of the used solvent temporarily in order to enhance its chiral purity. In addition, it is possible to enhance the yield of the salt by optionally cooling the precipitated salt before the filtering process. The amount of the optically active acid (dividing agent) is suitably 0.5 to 2.0 moles per one mole of the substance, preferably around the equivalent thereof. It is also possible to obtain a highly pure optically-active salt by optionally re-crystallizing the initially-obtained crystal in a solvent suitable for crystallization such as an alcohol. If necessary, the optically active compound of formula [1] can be obtained as a free form by conventionally treating the obtained salt with a base.

The present compound can be administered orally or parenterally in the medical use. Namely, the compound can be orally administered as a generally-used dosage form such as powder, granule, tablet, capsules, syrup, and suspension, or parenterally administered as an injection form such as solution, emulsion, and suspension thereof. And it can be rectally administered as a suppository. Furthermore, it can be intravesically administered as a solution. The above-mentioned drug form can be prepared by formulating the present compound with conventional additives such as carrier, excipient, binder, stabilizer, and diluent. In the case of injections, for example, acceptable buffer, solubilizer, and isotonic agent can be also used. In the case of the above-mentioned oral formulation or suppository, the present compound may be contained preferably in 0.1-70% (w/w) per the composition. The dosage and the frequency of administration depend on various conditions such as target disease, symptom, age and body weight of a subject, type of formulation, and manner of administration. In general, the present compound can be administered in a dosage of 0.1-2000 mg, preferably 1-200 mg per a day for an adult, and once to several times (e.g. twice to 4 times) a day.

The compounds of the invention are useful for treating psychosis, in more detail as follows.

The compounds of the invention exhibit high affinity for one or plural subtypes of various receptors, for example, dopaminergic receptor such as dopamine D₁ receptor, dopamine D₂ receptor, dopamine D₃ receptor and dopamine D₄ receptor; serotonergic receptor such as serotonin 5-HT_{1A} and serotonin 5-HT₂; and noradrenergic receptor such as α1 noradrenergic receptor and α2 noradrenergic receptor.

It has been well known that D2 receptor antagonistic action in a subtype of dopaminergic receptor is strongly correlated with psychotic effect (see: e.g. Seeman, Pharmacol. Rev., 32, 229 (1981)). And also, it has been reported that 5-HT₂ receptor antagonistic action in a subtype of serotonergic receptor is useful for antipsychotic effect (see: e.g. Janssen et al., J. Pharm. Exper. Ther., 244, 685 (1988)). Especially, D2 receptor antagonistic action can control positive symptoms of schizophrenia (e.g. hallucination, delusion), while 5-HT₂ receptor antagonistic action can contribute to improve negative symptoms of schizophrenia (e.g. indifference, social withdrawal). In addition, it has been suggested that 5-HT₂ receptor antagonistic action can decrease some side effects in the extrapyramidal tract which often arises in a maintenance therapy of schizophrenia using D2 receptor antagonist.

In addition, it has been recently suggested that D4 antagonistic action which is one of other dopaminergic receptor subtypes does not cause the side effects in the extrapyramidal tract which often arise in a maintenance therapy of schizophrenia (see, e.g. Seeman et al., Nature, 350, 610 (1991); Seeman at al., Nature, 358, 149 (1992)).

Further, it has been reported that antagonistic action of 5-HT_{1A} receptor which is a subtype of other serotonergic receptors is correlated with antianxiety (see: e.g. Titeler, Biochem, Pharmacol., 36, 3265 (1987)).

Accordingly, the compounds of the invention have psychotropic actions such as antipsychotic action, antianxiety, and antidepressive action, which are useful, for example, as a medicament for treating schizophrenia, senile insanity psychosis, bipolar disorder, neurosis, senile dementia and associated symptoms thereof, etc.

### EXAMPLE

### Example 1

### N-{[(1R,2R)-2-{[4-(1,2-benzisothiazol-3-yl)piperazin-1-yl]-methyl}cyclohexyl]methyl}-1,3-benzodioxole-5-carboxamide [Compound (6)]

### (A) Synthesis of Compound (3)

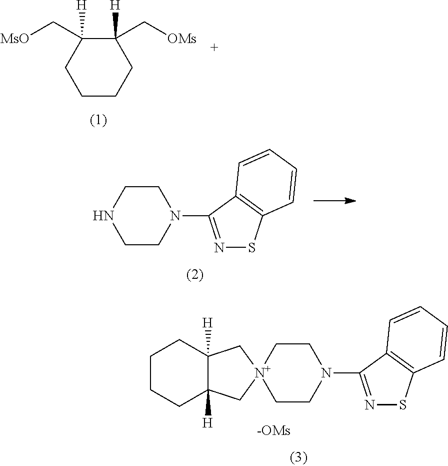

A mixture of (1R,2R)-cyclohexane-1,2-diyldimethanediyl dimethanesulfonate (1) (11.7 g, 38.9 mmol), 3-(piperazin-1-yl)-1,2-benzisothiazole (2) (7.76 g, 35.4 mmol), potassium carbonate (4.9 g, 35.4 mmol) and acetonitrile (200 ml) was refluxed for 20 hours. The mixture was filtrated at the hot state thereof, and the filtrate was concentrated to give Compound (3) (12 g, 28.3 mmol, yield: 80%).

### (B) Synthesis of Compound (4)

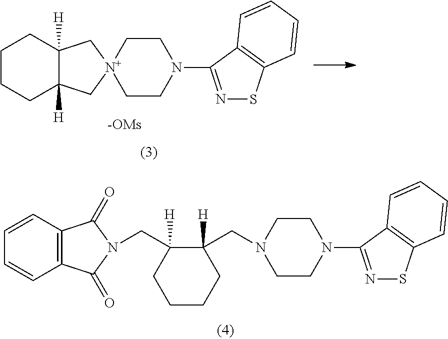

A mixture of Compound (3) (12 g, 28.3 mmol), potassium phthalimide (8.9 g, 48.1 mmol) and dimethylformamide (200 ml) was heated at 150° C. for 15 hours, and then ethyl acetate and saturated aqueous sodium bicarbonate were added to the mixture. The organic layer was washed with aqueous sodium bicarbonate and brine, dried and concentrated in vacuo to give 2-{[(1R,2R)-2-{[4-(1,2-benzisothiazol-3-yl)piperazin-1-yl]methyl}cyclohexyl]methyl}-1H-isoindole-1,3(2H)-dione [Compound (4)] (12.3 g, 26.0 mmol, yield: 92%).

MS (ESI+) (M+1, %); 475 (100).

### (C) Synthesis of Compound (5)

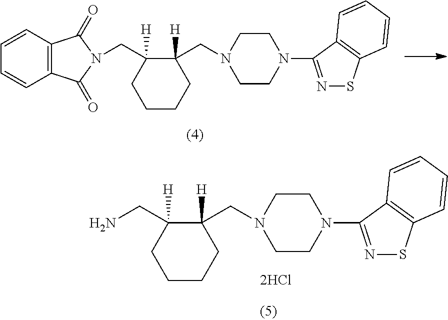

A mixture of Compound (4) (15 g, 28.3 mmol, crude), aqueous hydrazine (10 ml) and ethanol (300 ml) was refluxed for 30 minutes. The mixture was filtrated, and the filtrate was concentrated to give Compound (5) as a free base. The compound was treated with hydrochloric acid to give 1-[(1R,2R)-2-{[4-(1,2-benzisothiazol-3-yl)piperazin-1-yl]methyl}cyclohexyl]methanamine dihydrochloride [Compound (5)] (10.8 g, 26 mmol, yield: 92%).

MS (ESI+) (M+1, %); 345 (100).

### (D) Synthesis of Compound (6)

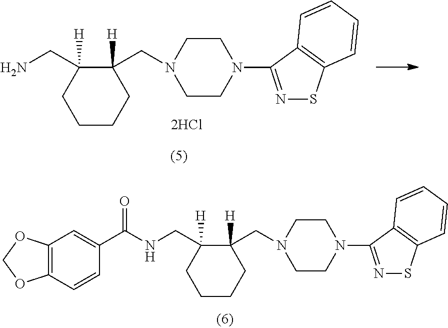

A mixture of Compound (5) (70 mg, 0.2 mmol), triethylamine (20 μl, 0.24 mmol), piperonyl chloride (47 mg, 0.26 mmol) and chloroform (3 ml) was stirred at room temperature for 7 hours, and then ethyl acetate and saturated aqueous sodium bicarbonate were added to the mixture. The organic layer was washed with aqueous sodium bicarbonate and brine, dried and concentrated. The resulting residue was purified by a silica gel chromatography to give N-{[1R,2R)-2-{[4-(1,2-benz-isothiazol-3-yl)piperazin-1-yl]methyl}cyclohexyl]methyl}-1,3-benzodioxole-5-carboxamide [Compound (6)] (93 mg, 0.19 mmol, yield: 93%).

MS (ESI+) (M+1, %); 493 (100), ¹H-NMR (300 MHz, CDCl₃) δ 7.86 (m, 2H), 7.40 (brm, 4H), 6.80 (dd, 1H, J=9.0, 6.0 Hz), 5.98 (s, 2H), 3.67 (m, 4H), 3.56 (m, 2H), 3.47 (m, 4H), 2.85 (m, 1H), 2.65 (m, 1H), 1.73 (m, 4H), 1.24 (brm, 6H).

### Example 2

### Synthesis of N-{[(1R,2R)-2-{[4-(1,2-benzisothiazol-3-yl)-piperazin-1-yl]methyl}cyclohexyl]methyl}biphenyl-3-sulfonamide (7)

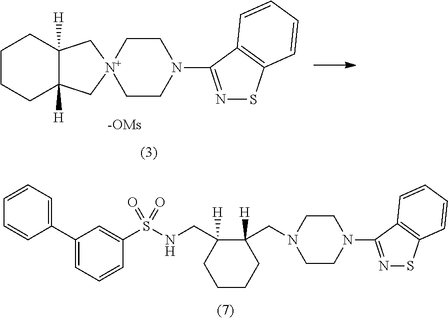

A mixture of Compound (3) (130 mg, 0.3 mmol) which is a synthetic intermediate in the above Example 1, 3-phenylbenzenesulfonyl amide (75 mg, 0.4 mmol), potassium carbonate (54 mg, 0.4 mmol), 18-crown-6-ether (5 mg, 18 μmol) and acetonitrile (2 ml) was refluxed for 15 hours, and then ethyl acetate and saturated aqueous sodium bicarbonate were added to the mixture. The organic layer was washed with aqueous sodium bicarbonate and brine, dried and concentrated in vacuo. The resulting residue was purified by a silica gel chromatography to give N-{[(1R,2R)-2-{[4-(1,2-benzisothiazol-3-yl)piperazin-1-yl]-methyl}cyclohexyl]methyl}biphenyl-3-sulfonamide [Compound (7)] (35 mg, 67 μmol, yield: 17%).

MS (ESI+) (M+1, %); 561 (100), ¹H-NMR (300 MHz, CDCl₃) δ 8.10 (m, 1H), 7.84 (m, 3H), 7.75 (m, 1H), 7.58 (m, 3H), 7.50 (m, 1H), 7.43 (m, 1H), 7.38 (m, 2H), 7.34 (m, 1H), 3.65 (m, 3H), 3.48 (m, 5H), 3.07 (dd, 1H, J=12.0, 5.0 Hz), 2.70 (brm, 3H), 1.62 (brm, 4H), 1.36 (m, 2H), 1.23 (m, 3H), 1.04 (m, 1H).

### Example 3

### N-{[(1S,2S)-2-{[4-(1,2-benzisothiazol-3-yl)piperazin-1-yl]-methyl}cyclopropyl]methyl}-1,3-benzodioxole-5-carboxamide [Compound (14)]

### (A) Synthesis of Compound (9)

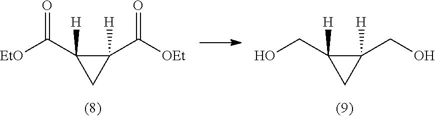

A mixture of diethyl cyclopropane-dicarboxylate (8) (6.5 g, 34.9 mmol) and tetrahydrofuran (24 ml) was added dropwise at ice temperature to a mixture of lithium aluminium hydride (2.4 g, 62.8 mmol) and tetrahydrofuran (50 ml), The mixture was stirred at room temperature for 2 hours and then cooled. Water (44 ml) and Celite (22 g) were added to the mixture in order, and the mixture was filtrated. The filtrate was washed with brine, dried, and concentrated in vacuo to give (1S,2S)-cyclopropane-1,2-diyldimethanol [Compound (9)] (1.36 g, 11.3 mmol, yield: 32%).

### (B) Synthesis of Compound (10)

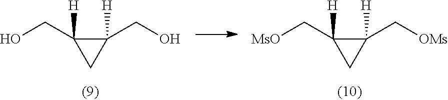

To a mixture of Compound (9) (1.36 g, 11.3 mmol), triethylamine (6.9 ml, 56.5 mmol) and chloroform (40 ml) was added methanesulfonyl chloride (4.1 g, 36.1 mmol) dropwise in ice temperature. The mixture was stirred at room temperature for two days, and then saturated aqueous sodium bicarbonate and ethyl acetate were added to the mixture. The organic layer was washed with aqueous sodium bicarbonate and brine, dried and concentrated in vacuo to give (1S,2S)-cyclopropane-1,2-diyldimethanediyl dimethane-sulfonate [Compound (10)] (2.9 g, 11.2 mmol, yield: 99%).

### (C) Synthesis of Compound (II)

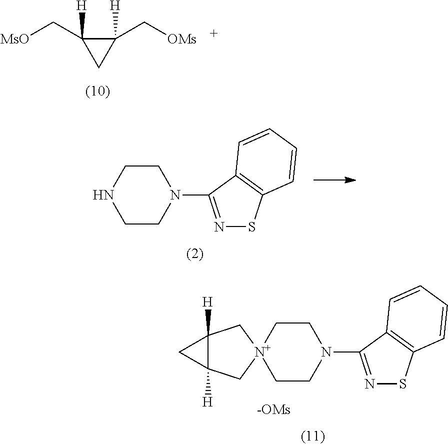

A mixture of Compound (10) (2.9 g, 11.2 mmol), 3-(piperazin-1-yl)-1,2-benzisothiazole (2) (1.9 g, 8.9 mmol), potassium carbonate (1.2 g, 8.9 mmol) and acetonitrile (50 ml) was refluxed for 20 minutes. The mixture was filtrated at the hot state thereof, and the filtrate was concentrated to give Compound (II) (4.2 g, 11.0 mmol, yield: 98%).

### (D) Synthesis of Compound (12)

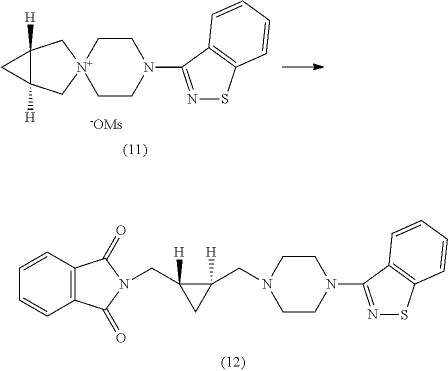

A mixture of Compound (II) (4.2 g, 11.0 mmol), potassium phthalimide (3.5 g, 18.9 mmol) and dimethyl-formamide (40 ml) was heated at 180° C. for 4 hours, and then ethyl acetate and saturated aqueous sodium bicarbonate were added to the mixture. The organic layer was washed with aqueous sodium bicarbonate and brine, dried and concentrated in vacuo to give 2-{[(1S,2S)-2-{[4-(1,2-benzisothiazol-3-yl)piperazin-1-yl]methyl}cyclopropyl]-methyl}-1H-isoindole-1,3(2H)-dione [Compound (12)] (3.4 g, 7.8 mmol, yield: 71%).

MS (ESI+) (M+1, %); 433 (100).

### (E) Synthesis of Compound (13)

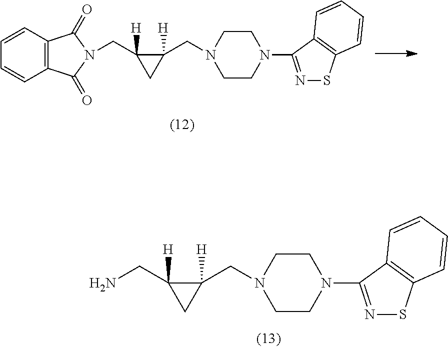

A mixture of Compound (12) (3.4 g, 7.8 mmol), aqueous hydrazine (12 ml) and ethanol (30 ml) was refluxed for 2 hours. The mixture was filtrated, and the filtrate was concentrated to give 1-[(1S,2S)-2-{[4-(1,2-benzisothiazol-3-yl)piperazin-1-yl]methyl}cyclopropyl]methanamine [Compound (13)] (2.1 g, 70 mmol, yield: 91%).

MS (ESI+) (M+1, %); 303 (100).

### (F) Synthesis of Compound (14)

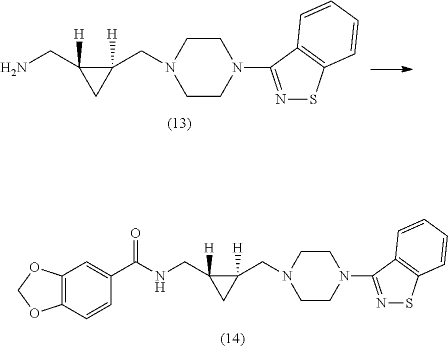

A mixture of Compound (13) (24 mg, 78 μmol), triethylamine (350 μl), piperonyl chloride (29 mg, 0.15 mmol) and chloroform (3 ml) was stirred at room temperature for 7 hours, and then ethyl acetate and saturated aqueous sodium bicarbonate were added to the mixture. The organic layer was washed with aqueous sodium bicarbonate and brine, dried and concentrated in vacuo. The resulting residue was purified by a silica gel chromatography to give N-{[(1S,2S)-2-{[4-(1,2-benzisothiazol-3-yl)piperazin-1-yl]-methyl}cyclopropyl]methyl}-1,3-benzodioxole-5-carboxamide [Compound (14)] (32 mg, 72 μmol, yield: 91%).

MS (ESI+) (M+1, %); 451 (100), ¹H-NMR (300 MHz, CDCl₃) δ 7.84 (dd, 2H, J=15.0, 6.0 Hz), 7.47 (t, 1H, J=7.5 Hz), 7.34 (m, 4H), 6.80 (d, 1H, J=6.0 Hz), 5.97 (s, 2H), 3.57 (m, 4H), 3.36 (m, 2H), 2.77 (m, 4H), 2.55 (dd, 1H, J=12.0, 6.0 Hz), 2.28 (dd, 1H, J=12.0, 6.0 Hz), 0.99 (m, 2H), 0.60 (dt, 1H, J=9.0, 6.0 Hz), 0.47 (dt, 1H, J=9.0, 6.0 Hz).

### Example 4

### N-{[(1S,2S)-2-{[4-(1,2-benzisothiazol-3-yl)piperazin-1-yl]-methyl}cyclopentyl]methyl}-1,3-benzodioxole-5-carboxamide [Compound (21)]

### (A) Synthesis of Compound (16)

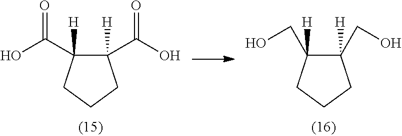

A mixture of cyclopentane-dicarboxylic acid (15) (2.0 g, 12.6 mmol) and tetrahydrofuran (20 ml) was added dropwise at room temperature to a mixture of lithium aluminium hydride (1.4 g, 38.0 mmol) and tetrahydrofuran (40 ml). The mixture was stirred at 70° C. for 8 hours and then cooled. Water (30 ml) and Celite (25 g) were added to the mixture in order, and the mixture was filtrated. The filtrate was washed with brine, dried, and concentrated in vacuo to give (1S,2S)-cyclopentane-1,2-diyldimethanol [Compound (16)] (1.6 g, 12.6 mmol, quantitative).

### (B) Synthesis of Compound (17)

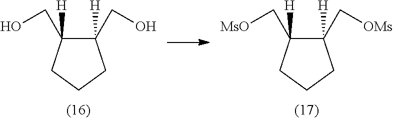

To a mixture of Compound (16) (1.6 g, 12.6 mmol), triethylamine (8.0 ml, 64.5 mmol) and chloroform (20 ml) was added methanesulfonyl chloride (4.7 g, 41.3 mmol) dropwise in ice temperature. The mixture was stirred at room temperature for 3 days, and then saturated aqueous sodium bicarbonate and ethyl acetate were added to the mixture. The organic layer was washed with aqueous sodium bicarbonate and brine, dried and concentrated in vacuo to give (1S,2S)-cyclopentane-1,2-diyldimethanediyl dimethane-sulfonate [Compound (17)] (3.6 g, 12.6 mmol, quantitative).

### (C) Synthesis of Compound (18)

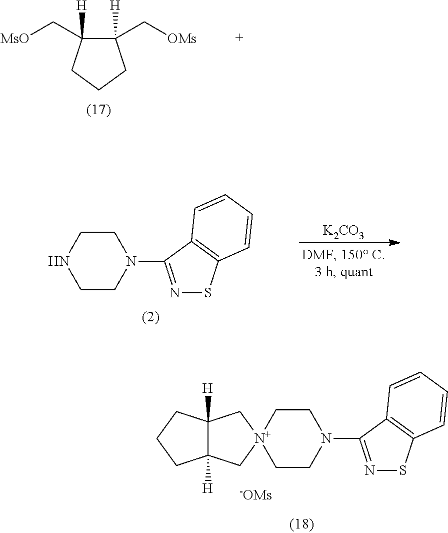

A mixture of Compound (17) (2.0 g, 6.7 mmol), 3-(piperazin-1-yl)-1,2-benzisothiazole (2) (1.4 g, 6.4 mmol), potassium carbonate (0.8 g, 6.5 mmol) and dimethylformamide (10 ml) was stirred at 150° C. for 3 hours. The mixture was filtrated at the hot state thereof, and the filtrate was concentrated to give Compound (18) (2.7 g, 6.7 mmol, quantitative).

### (D) Synthesis of Compound (19)

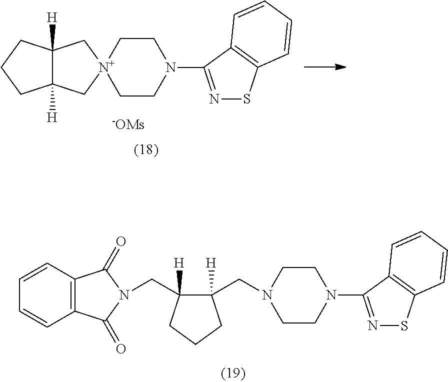

A mixture of Compound (18) (2.4 g, 6.0 mmol), potassium phthalimide (1.3 g, 7.2 mmol) and dimethyl-formamide (30 ml) was refluxed for 4 hours, and then ethyl acetate and saturated aqueous sodium bicarbonate were added to the mixture. The organic layer was washed with aqueous sodium bicarbonate and brine, dried and concentrated in vacuo. The resulting residue was purified by a silica gel chromatography to give 2-{[(1S,2S)-2-{[4-(1,2-benz-isothiazol-3-yl)piperazin-1-yl]methyl}cyclopentyl]methyl}-1H-isoindole-1,3(2H)-dione [Compound (19)] (2.2 g, 4.8 mmol, yield: 81%).

MS (ESI+) (M+1, %); 461 (100).

### (E) Synthesis of Compound (20)

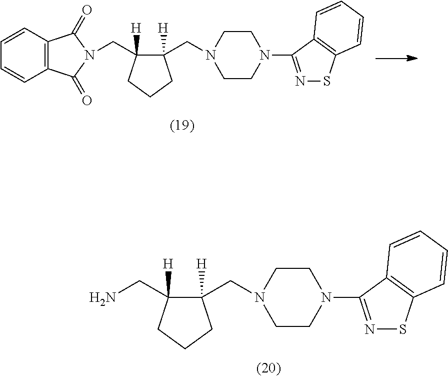

A mixture of Compound (19) (2.2 g, 4.8 mmol), aqueous hydrazine (2 ml) and ethanol (60 ml) was refluxed for 2 hours, and then filtrated. Ethyl acetate and water were added to the filtrate. The organic layer was washed with brine, dried and concentrated in vacuo to give 1-[(1S,2S)-2-{[4-(1,2-benzisothiazol-3-yl)piperazin-1-yl]methyl}cyclo-pentyl]methanamine [Compound (20)] (0.7 g, 2.2 mmol, yield: 46%).

MS (ESI+) (M+1, %); 331 (100).

### (F) Synthesis of Compound (21)

### Synthesis of N-{[(1S,2S)-2-{[4-(1,2-benzisothiazol-3-yl)-piperazin-1-yl]methyl}cyclopentyl]methyl}-1,3-benzodioxole-5-carboxamide (21)

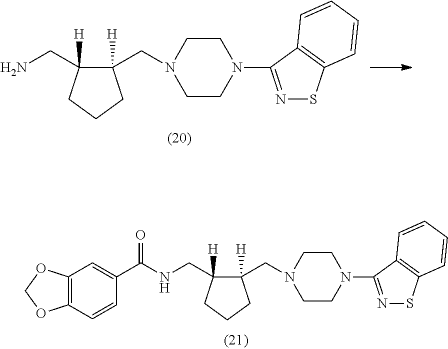

A mixture of 1-[(1S,2S)-2-{[4-(1,2-benzisothiazol-3-yl)piperazin-1-yl]methyl}cyclopentyl]methanamine (20) (28 mg, 84 μmol), triethylamine (200 μl), piperonyl chloride (29 mg, 0.15 mmol) and chloroform was stirred at room temperature for 7 hours, and then ethyl acetate and saturated aqueous sodium bicarbonate were added to the mixture. The organic layer was washed with aqueous sodium bicarbonate and brine, dried and concentrated in vacuo. The resulting residue was purified by a silica gel chromatography to give N-{[(1S,2S)-2-{[4-(1,2-benz-isothiazol-3-yl)piperazin-1-yl]methyl}cyclopentyl]methyl}-1,3-benzodioxole-5-carboxamide (21) (34 mg, 71 μmol, yield: 85%).

MS (ESI+) (M+1, %); 479 (100), ¹H-NMR (300 MHz, CDCl₃) δ 7.85 (m, 2H), 7.48 (m, 1H), 7.34 (brm, 3H), 7.15 (m, 1H), 3.79 (m, 1H), 3.67 (1H, m), 3.56 (m, 5H), 3.51 (s, 2H), 3.39 (m, 1H), 2.87 (m, 2H), 2.66 (m, 1H), 2.44 (m, 1H), 1.82 (m, 3H), 1.56 (m, 3H), 1.28 (brm, 2H).

### Examples 5-243

The following compounds were prepared according to the processes in Examples 1 to 4.

| | | | MS (ESI+) |
|---|---|---|---|
| Example | | Molecular | (M + 1, peak |
| No. | Structure | Weight | intension) |
| 5 | 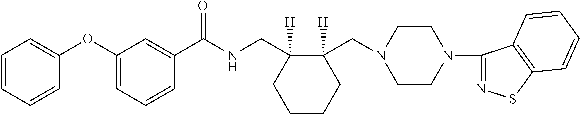 | 540.73 | 541 (100) |
| 6 | 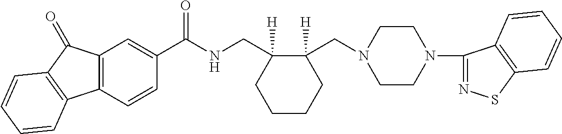 | 550.73 | 551 (100) |
| 7 | 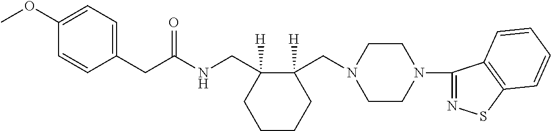 | 492.69 | 493 (100) |
| 8 | 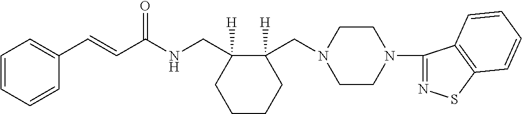 | 474.67 | 475 (100) |
| 9 | 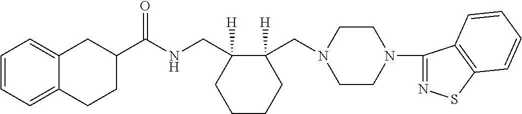 | 502.73 | 503 (100) |
| 10 | 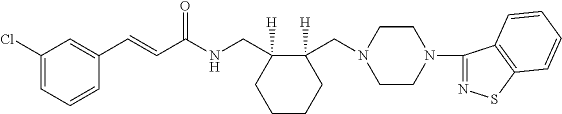 | 509.12 | 510 (100) |
| 11 | 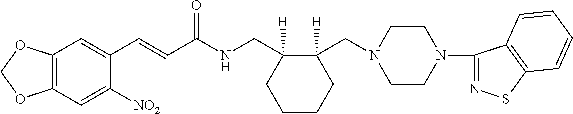 | 563.68 | 564 (100) |
| 12 | 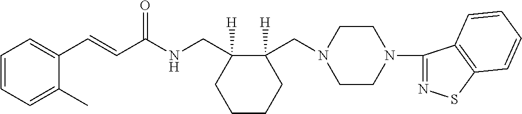 | 488.70 | 489 (100) |
| 13 | 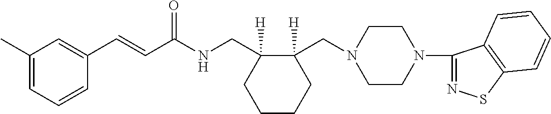 | 488.70 | 489 (100) |
| 14 | 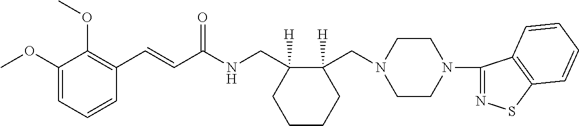 | 534.73 | 535 (100) |
| 15 | 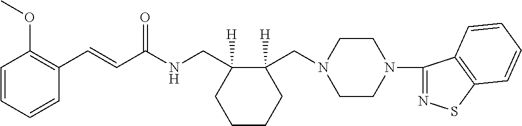 | 504.70 | 505 (100) |
| 16 | 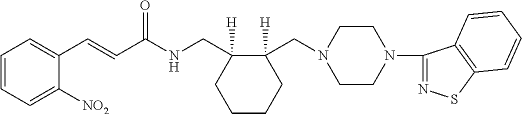 | 519.67 | 520 (100) |
| 17 | 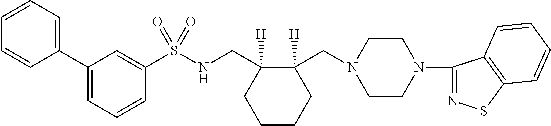 | 560.79 | 561 (100) |
| 18 | 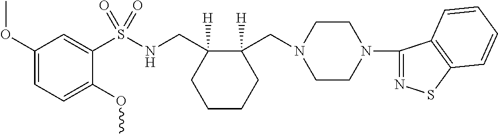 | 544.74 | 545 (100) |
| 19 | 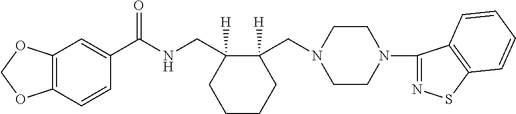 | 492.65 | 493 (100) |
| 20 | 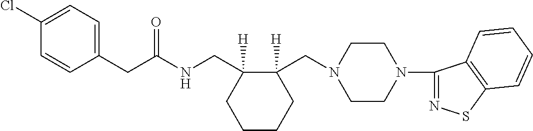 | 497.11 | 498 (100) |
| 21 | 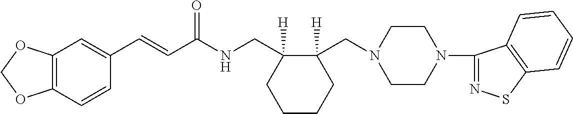 | 518.68 | 519 (100) |
| 22 | 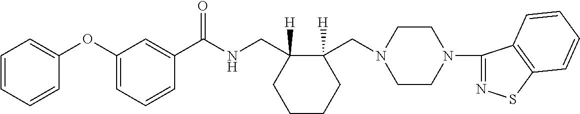 | 540.73 | 541 (100) |
| 23 | 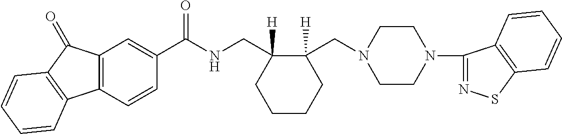 | 550.73 | 551 (100) |
| 24 | 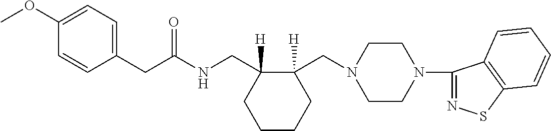 | 492.69 | 493 (100) |
| 25 | 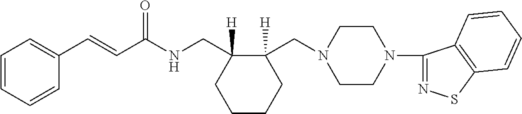 | 474.67 | 475 (100) |
| 26 | 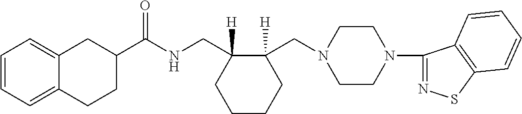 | 502.73 | 503 (100) |
| 27 | 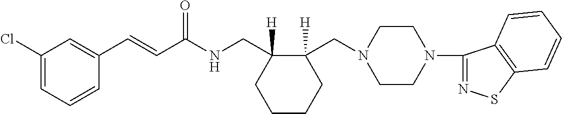 | 509.12 | 510 (100) |
| 28 | 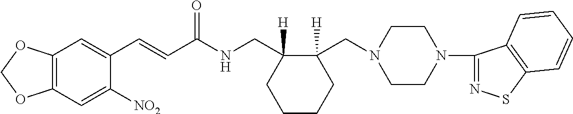 | 563.68 | 564 (100) |
| 29 | 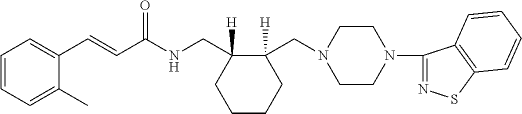 | 488.70 | 489 (100) |
| 30 | 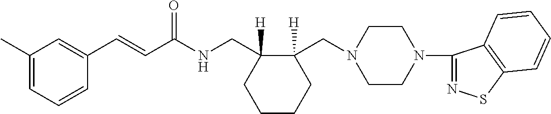 | 488.70 | 489 (100) |
| 31 | 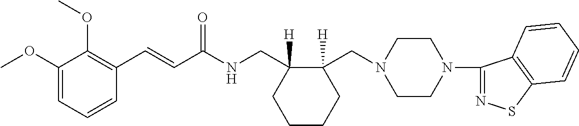 | 534.73 | 535 (100) |
| 32 | 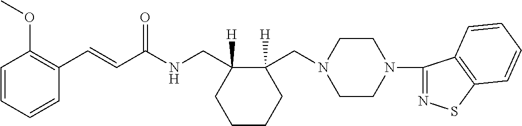 | 504.70 | 505 (100) |
| 33 | 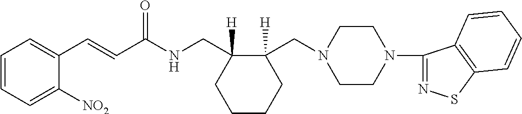 | 519.67 | 520 (100) |
| 34 | 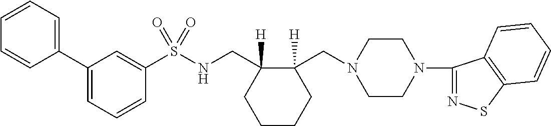 | 560.79 | 561 (100) |
| 35 | 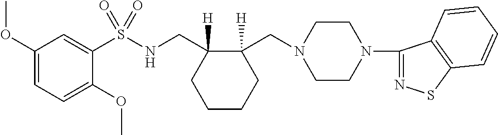 | 544.74 | 545 (100) |
| 36 | 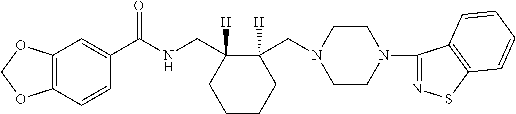 | 492.65 | 493 (100) |
| 37 | 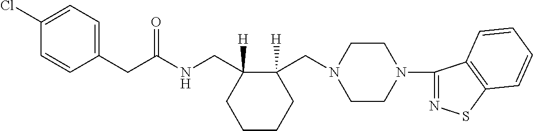 | 497.11 | 498 (100) |
| 38 | 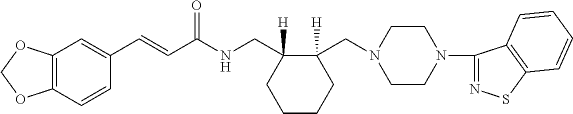 | 518.68 | 519 (100) |
| 39 | 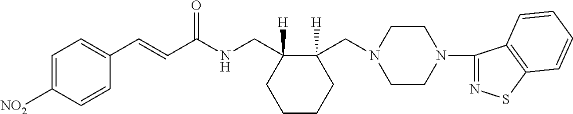 | 519.67 | 520 (100) |
| 40 | 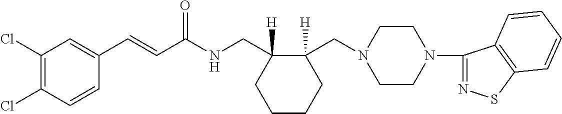 | 543.56 | 544 (100) |
| 41 | 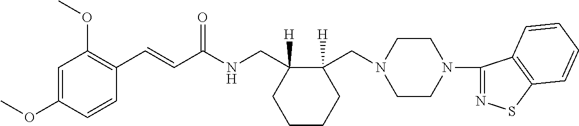 | 534.73 | 535 (100) |
| 42 | 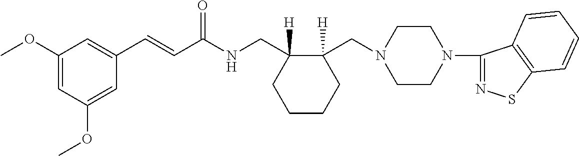 | 534.73 | 535 (100) |
| 43 | 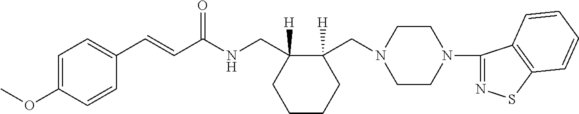 | 504.70 | 505 (100) |
| 44 | 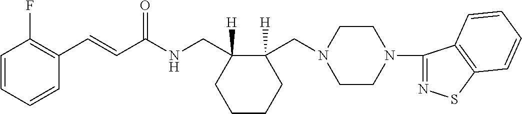 | 492.66 | 493 (100) |
| 45 | 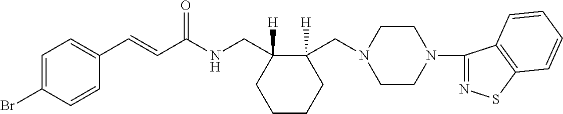 | 553.57 | 554 (100) |
| 46 | 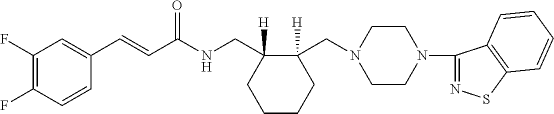 | 510.65 | 511 (100) |
| 47 | 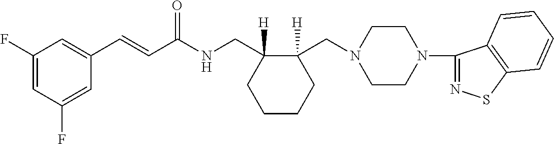 | 510.65 | 511 (100) |
| 48 | 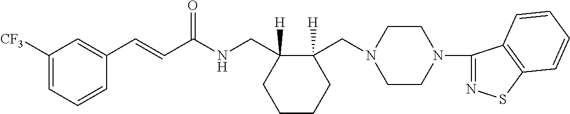 | 542.67 | 543 (100) |
| 49 | 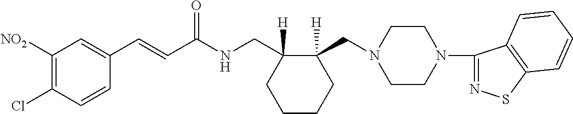 | 554.12 | 555 (100) |
| 50 | 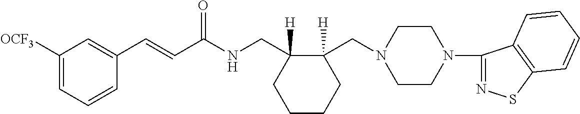 | 558.67 | 559 (100) |
| 51 | 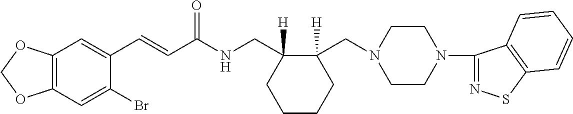 | 597.58 | 598 (100) |
| 52 | 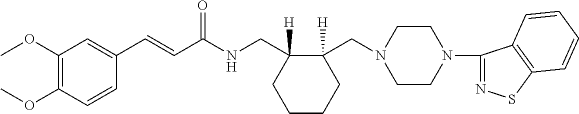 | 534.73 | 535 (100) |
| 53 | 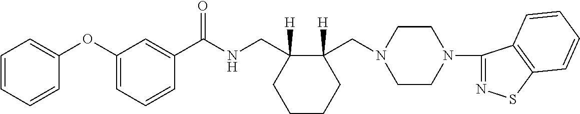 | 540.73 | 541 (100) |
| 54 | 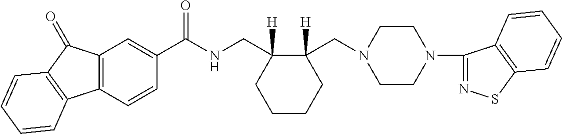 | 550.73 | 551 (100) |
| 55 | 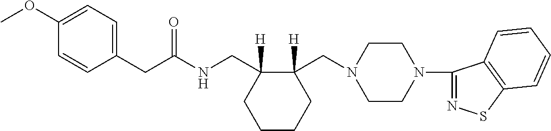 | 492.69 | 493 (100) |
| 56 | 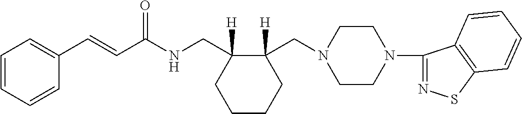 | 474.67 | 475 (100) |
| 57 | 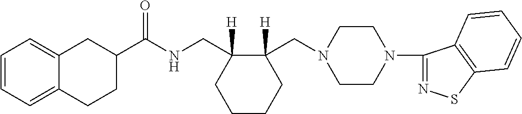 | 502.73 | 503 (100) |
| 58 | 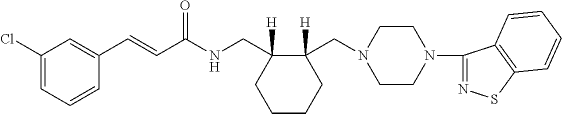 | 509.12 | 510 (100) |
| 59 | 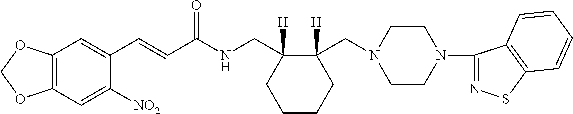 | 563.68 | 564 (100) |
| 60 | 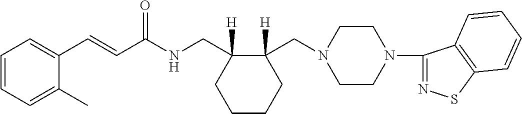 | 488.70 | 489 (100) |
| 61 | 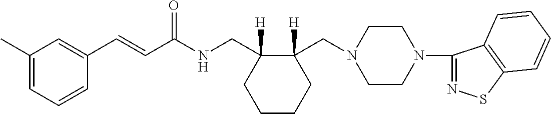 | 488.70 | 489 (100) |
| 62 | 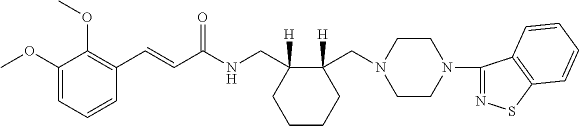 | 534.73 | 535 (100) |
| 63 | 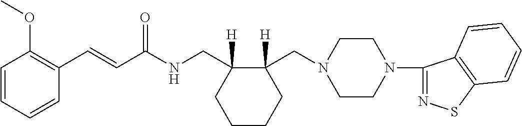 | 504.70 | 505 (100) |
| 64 | 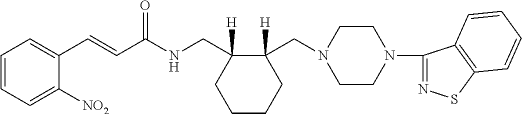 | 519.67 | 520 (100) |
| 65 | 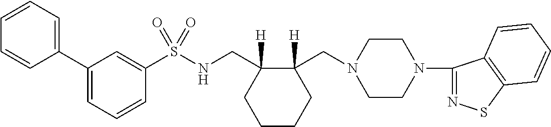 | 560.79 | 561 (100) |
| 66 | 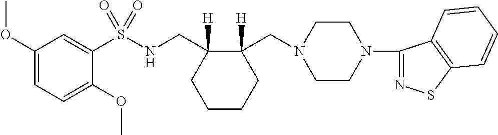 | 544.74 | 545 (100) |
| 67 | 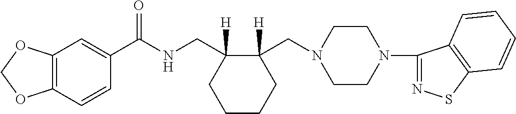 | 492.65 | 493 (100) |
| 68 | 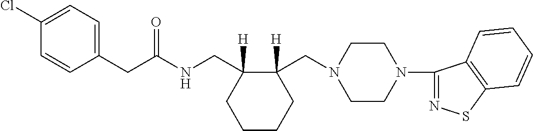 | 497.11 | 498 (100) |
| 69 | 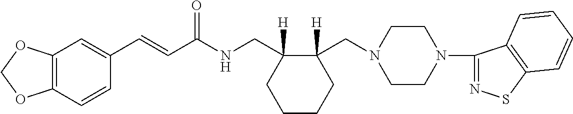 | 518.68 | 519 (100) |
| 70 | 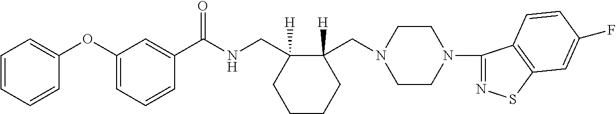 | 558.72 | 559 (100) |
| 71 | 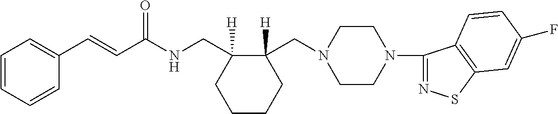 | 492.66 | 493 (100) |
| 72 | 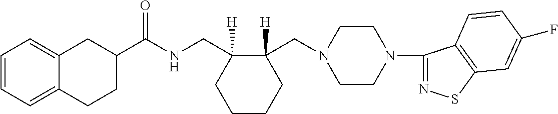 | 520.72 | 521 (100) |
| 73 | 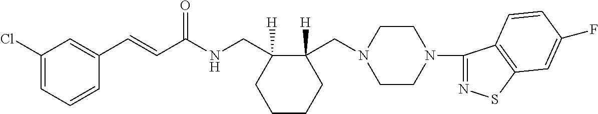 | 527.11 | 528 (100) |
| 74 | 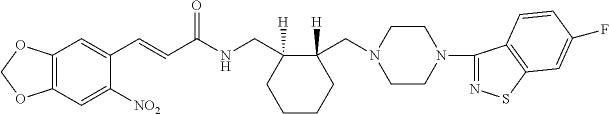 | 581.67 | 582 (100) |
| 75 | 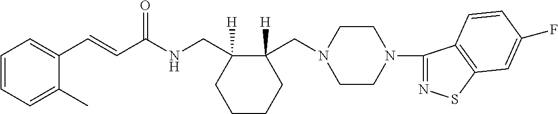 | 506.69 | 507 (100) |
| 76 | 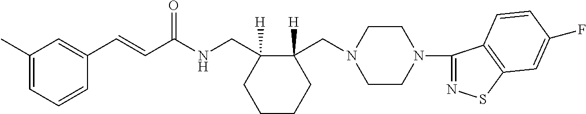 | 506.69 | 507 (100) |
| 77 | 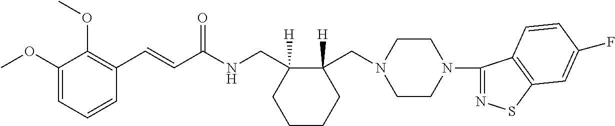 | 552.72 | 553 (100) |
| 78 | 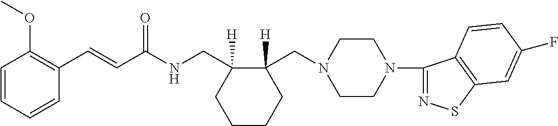 | 522.69 | 523 (100) |
| 79 | 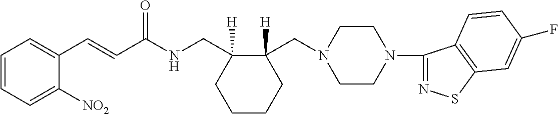 | 537.66 | 538 (100) |
| 80 | 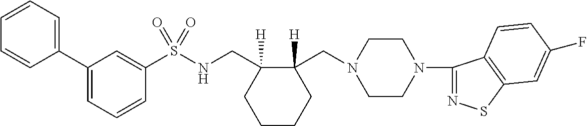 | 578.78 | 579 (100) |
| 81 | 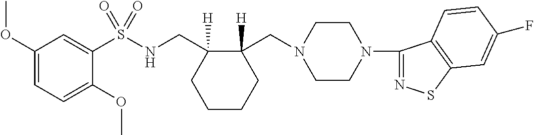 | 562.73 | 563 (100) |
| 82 | 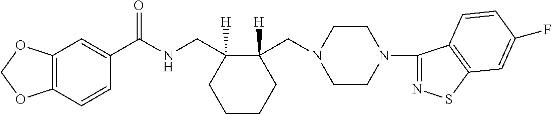 | 510.64 | 511 (100) |
| 83 | 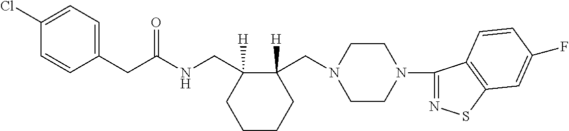 | 515.10 | 516 (100) |
| 84 | 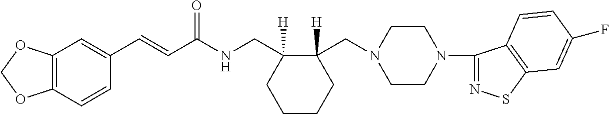 | 536.67 | 537 (100) |
| 85 | 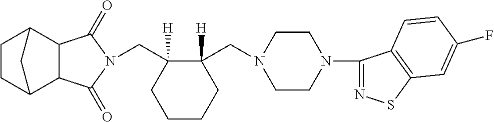 | 510.68 | 511 (100) |
| 86 | 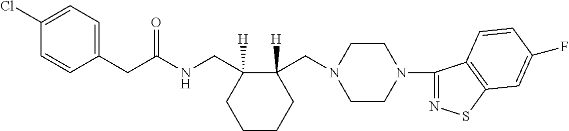 | 515.10 | 516 (100) |
| 87 | 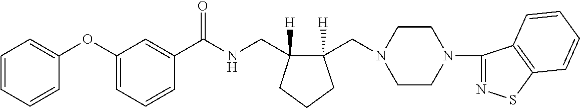 | 526.71 | 527 (100) |
| 88 | 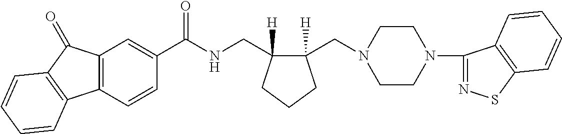 | 536.70 | 537 (100) |
| 89 | 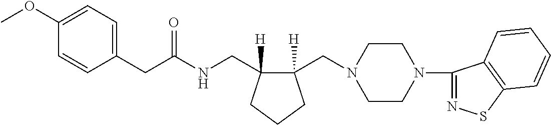 | 478.66 | 479 (100) |
| 90 | 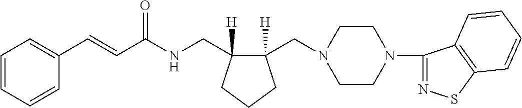 | 460.65 | 461 (100) |
| 91 | 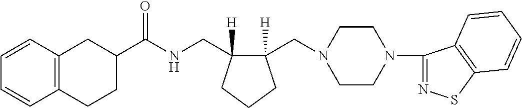 | 488.70 | 489 (100) |
| 92 | 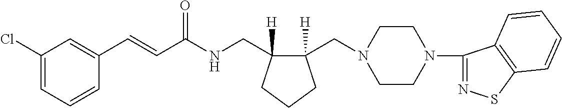 | 495.09 | 496 (100) |
| 93 | 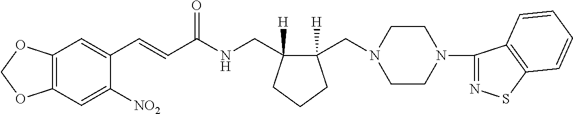 | 549.65 | 550 (100) |
| 94 | 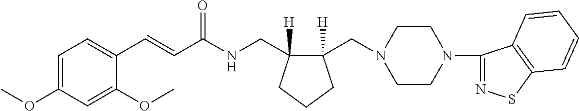 | 520.70 | 521 (100) |
| 95 | 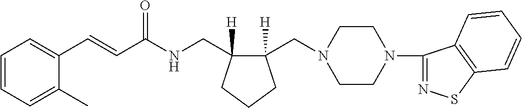 | 474.67 | 475 (100) |
| 96 | 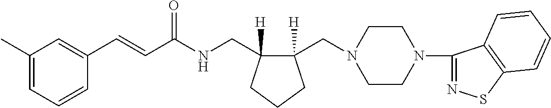 | 474.67 | 475 (100) |
| 97 | 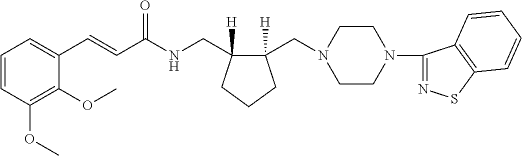 | 520.70 | 521 (100) |
| 98 | 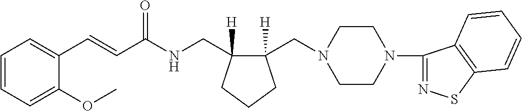 | 490.67 | 491 (100) |
| 99 | 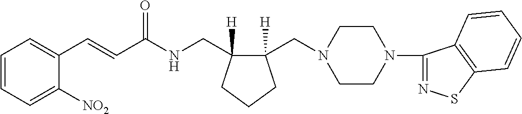 | 505.64 | 506 (100) |
| 100 | 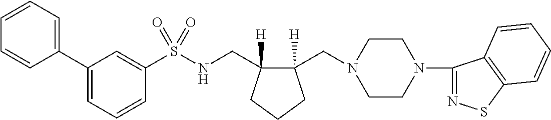 | 546.76 | 547 (100) |
| 101 | 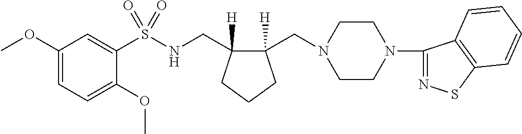 | 530.71 | 531 (100) |
| 102 | 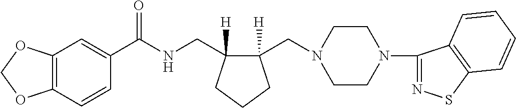 | 478.62 | 479 (100) |
| 103 | 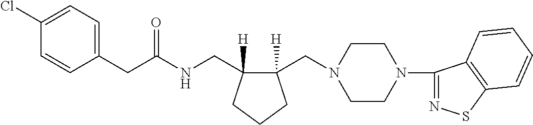 | 483.08 | 484 (100) |
| 104 | 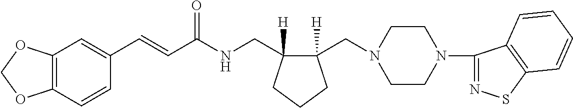 | 504.66 | 505 (100) |
| 105 | 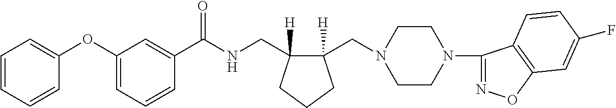 | 527.64 | 528 (100) |
| 106 | 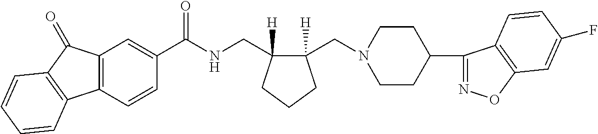 | 537.64 | 538 (100) |
| 107 | 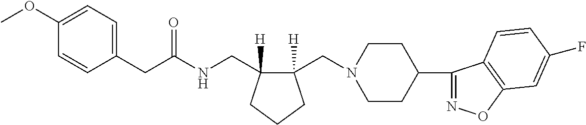 | 479.60 | 480 (100) |
| 108 | 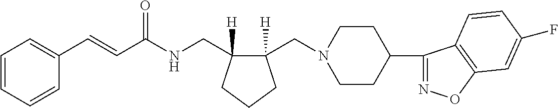 | 461.58 | 462 (100) |
| 109 | 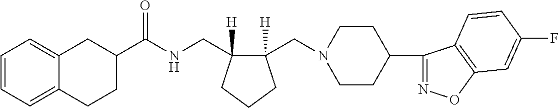 | 489.64 | 490 (100) |
| 110 | 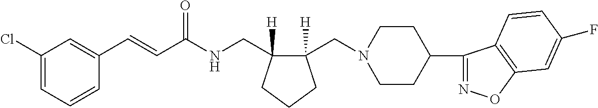 | 496.03 | 497 (100) |
| 111 | 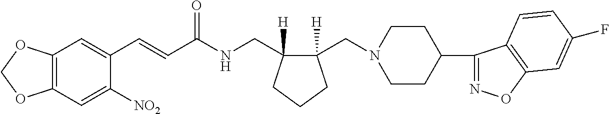 | 550.59 | 551 (100) |
| 112 | 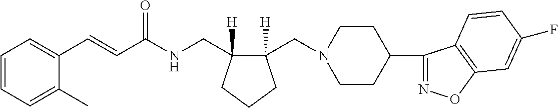 | 475.61 | 476 (100) |
| 113 | 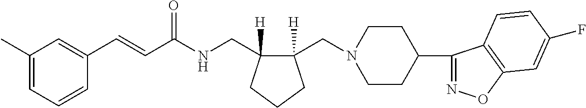 | 475.61 | 476 (100) |
| 114 | 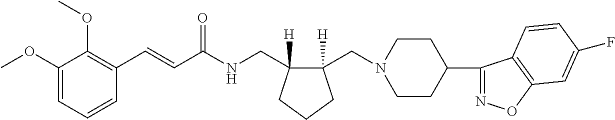 | 521.64 | 522 (100) |
| 115 | 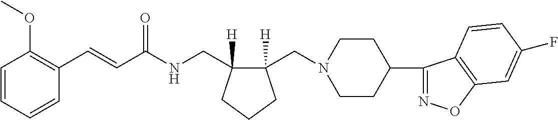 | 491.61 | 492 (100) |
| 116 | 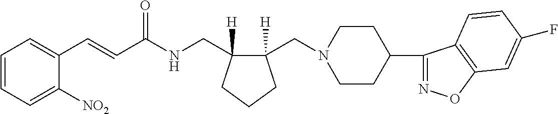 | 506.58 | 507 (100) |
| 117 | 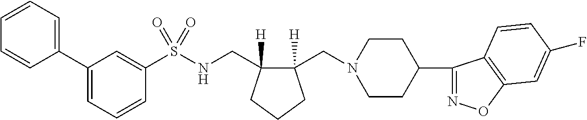 | 547.70 | 548 (100) |
| 118 | 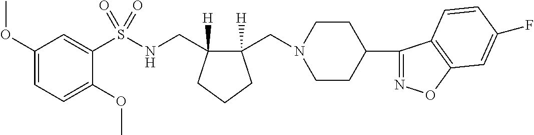 | 531.65 | 532 (100) |
| 119 | 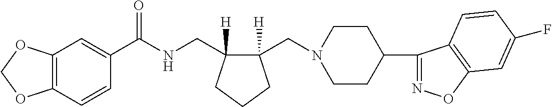 | 479.56 | 480 (100) |
| 120 | 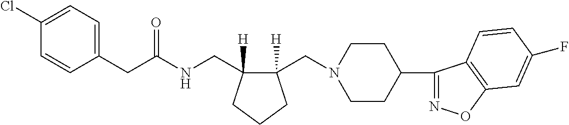 | 484.02 | 485 (100) |
| 121 | 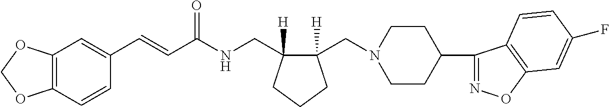 | 505.59 | 506 (100) |
| 122 | 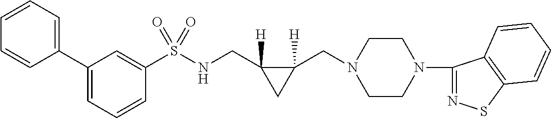 | 518.70 | 519 (100) |
| 123 | 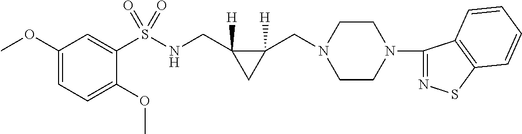 | 502.66 | 503 (100) |
| 124 | 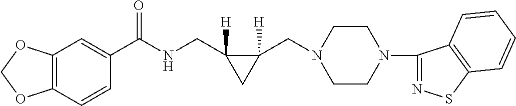 | 450.56 | 451 (100) |
| 125 | 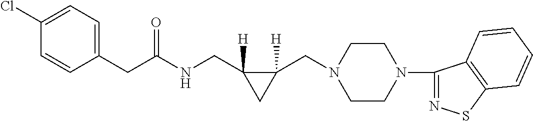 | 455.03 | 456 (100) |
| 126 | 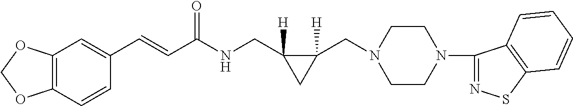 | 476.60 | 477 (100) |
| 127 | 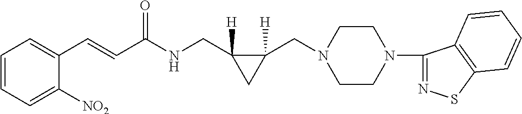 | 477.59 | 478 (100) |
| 128 | 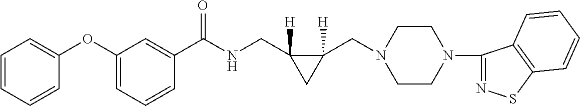 | 498.65 | 499 (100) |
| 129 | 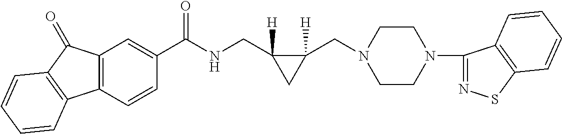 | 508.65 | 509 (100) |
| 130 | 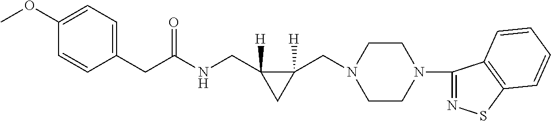 | 450.61 | 451 (100) |
| 131 | 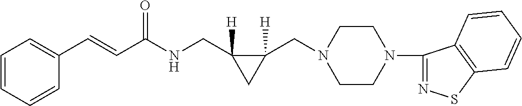 | 432.59 | 433 (100) |
| 132 | 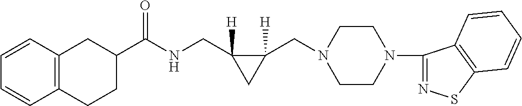 | 460.65 | 461 (100) |
| 133 | 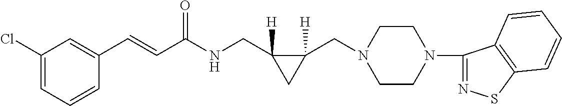 | 467.04 | 468 (100) |
| 134 | 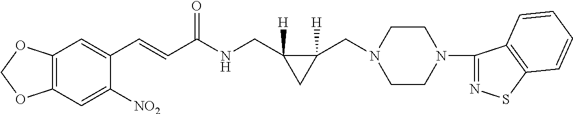 | 521.60 | 522 (100) |
| 135 | 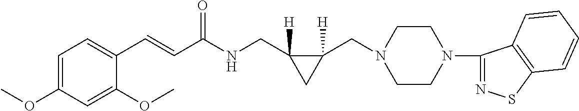 | 492.65 | 493 (100) |
| 136 | 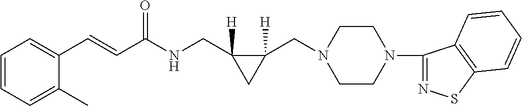 | 446.62 | 447 (100) |
| 137 | 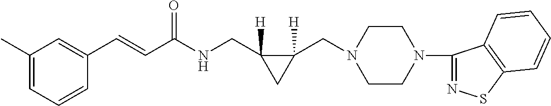 | 446.62 | 447 (100) |
| 138 | 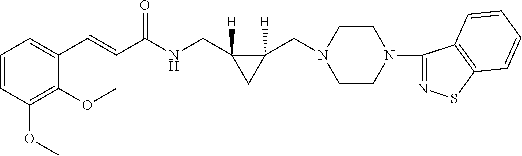 | 492.65 | 493 (100) |
| 139 | 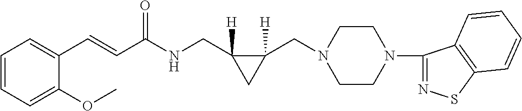 | 462.62 | 463 (100) |
| 140 | 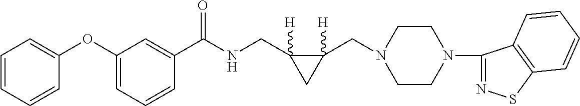 | 498.65 | 499 (100) |
| 141 | 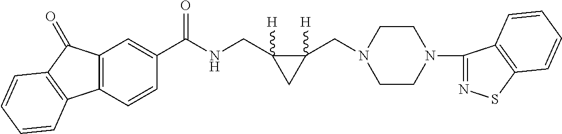 | 508.65 | 509 (100) |
| 142 | 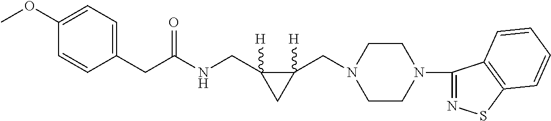 | 450.61 | 451 (100) |
| 143 | 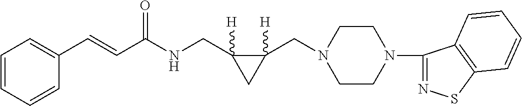 | 432.59 | 433 (100) |
| 144 | 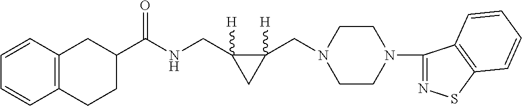 | 460.65 | 461 (100) |
| 145 | 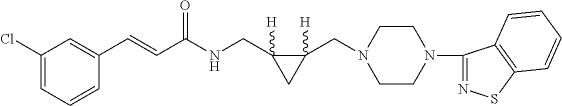 | 467.04 | 468 (100) |
| 146 | 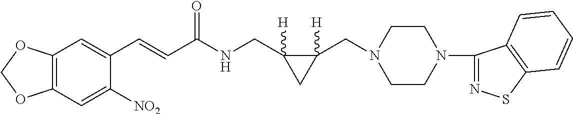 | 521.60 | 522 (100) |
| 147 | 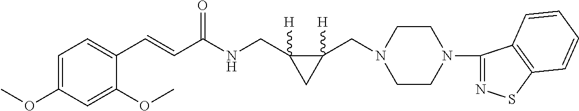 | 492.65 | 493 (100) |
| 148 | 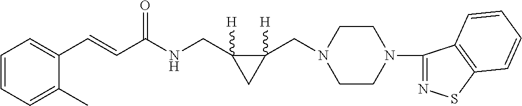 | 446.62 | 447 (100) |
| 149 | 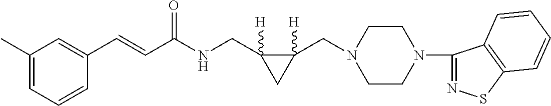 | 446.62 | 447 (100) |
| 150 | 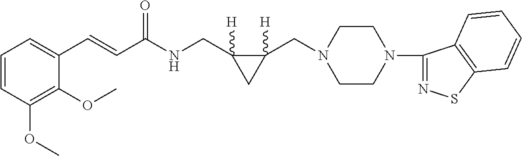 | 492.65 | 493 (100) |
| 151 | 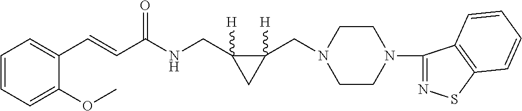 | 462.62 | 463 (100) |
| 152 | 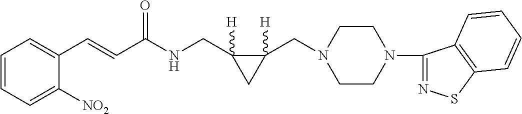 | 477.59 | 478 (100) |
| 153 | 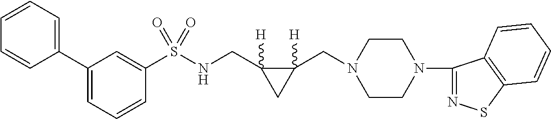 | 518.70 | 519 (100) |
| 154 | 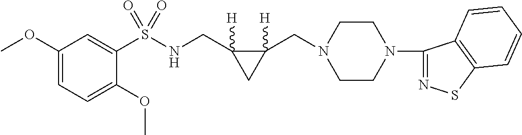 | 502.66 | 503 (100) |
| 155 | 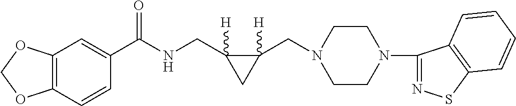 | 450.56 | 451 (100) |
| 156 | 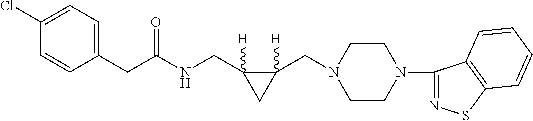 | 455.03 | 456 (100) |
| 157 | 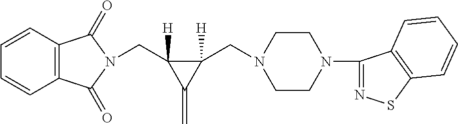 | 444.56 | 445 (100) |
| 158 | 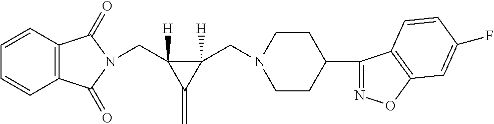 | 445.50 | 446 (100) |
| 159 | 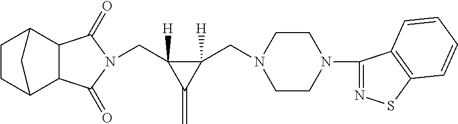 | 462.62 | 463 (100) |
| 160 | 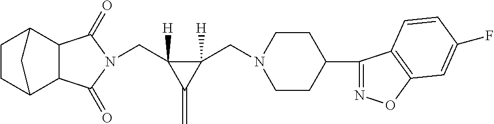 | 463.56 | 464 (100) |
| 161 | 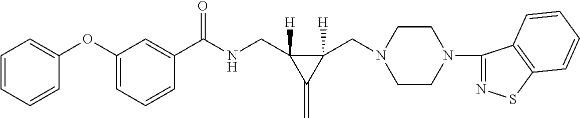 | 510.66 | 511 (100) |
| 162 | 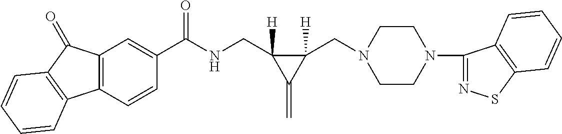 | 520.66 | 521 (100) |
| 163 | 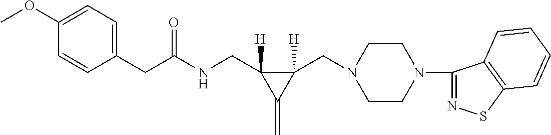 | 462.62 | 463 (100) |
| 164 | 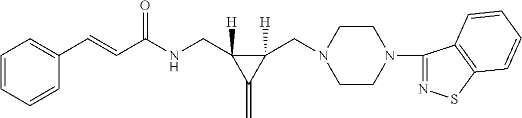 | 444.60 | 445 (100) |
| 165 | 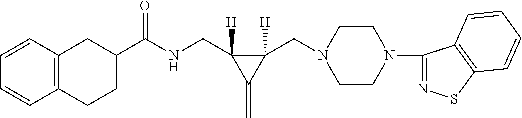 | 472.66 | 473 (100) |
| 166 | 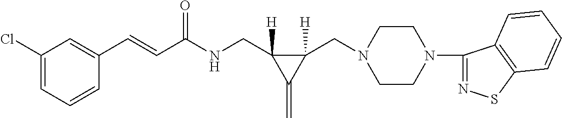 | 479.05 | 480 (100) |
| 167 | 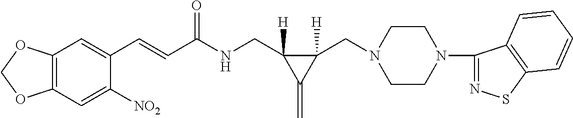 | 533.61 | 534 (100) |
| 168 | 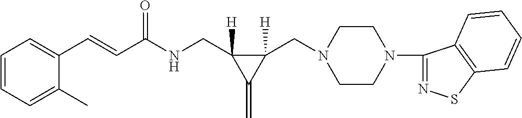 | 458.63 | 459 (100) |
| 169 | 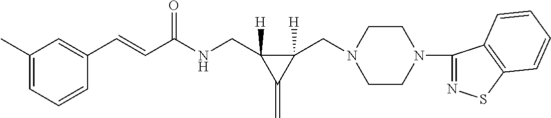 | 458.63 | 459 (100) |
| 170 | 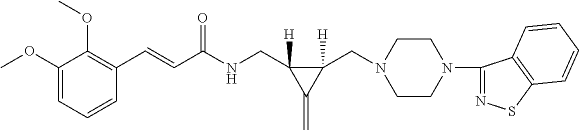 | 504.66 | 505 (100) |
| 171 | 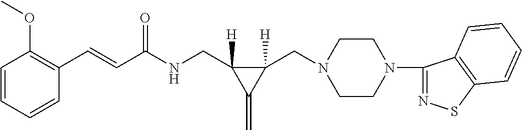 | 474.63 | 475 (100) |
| 172 | 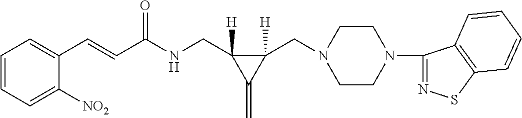 | 489.60 | 490 (100) |
| 173 | 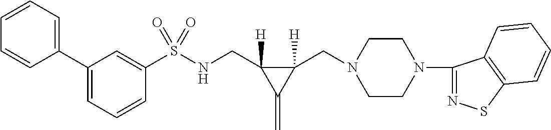 | 530.72 | 531 (100) |
| 174 | 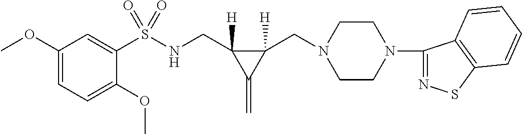 | 514.67 | 515 (100) |
| 175 | 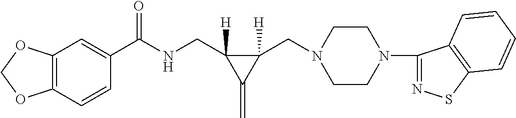 | 462.57 | 463 (100) |
| 176 | 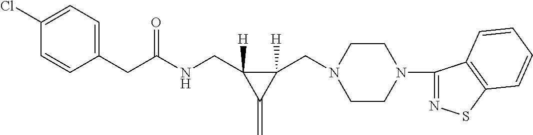 | 467.04 | 468 (100) |
| 177 | 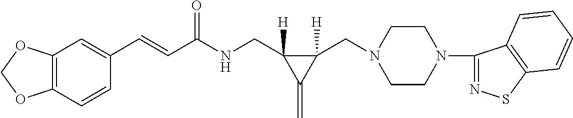 | 488.61 | 489 (100) |
| 178 | 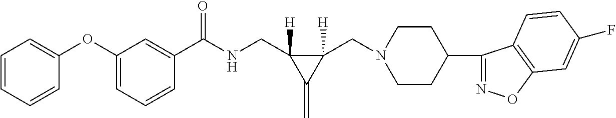 | 511.60 | 512 (100) |
| 179 | 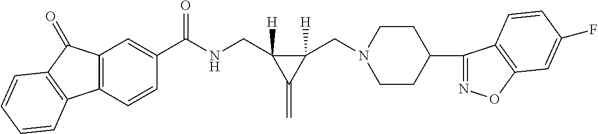 | 521.60 | 522 (100) |
| 180 | 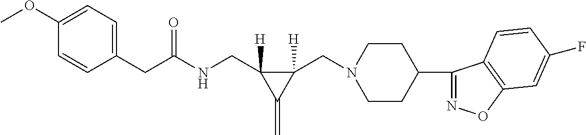 | 463.56 | 464 (100) |
| 181 | 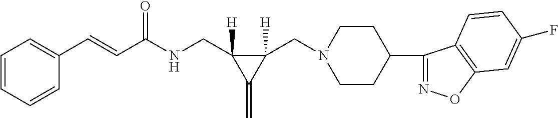 | 445.54 | 446 (100) |
| 182 | 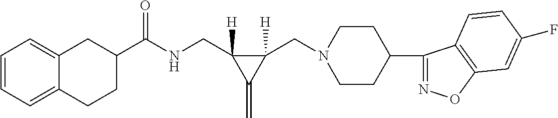 | 473.60 | 474 (100) |
| 183 | 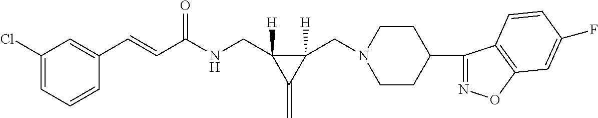 | 479.99 | 480 (100) |
| 184 | 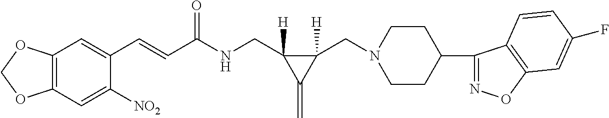 | 534.55 | 535 (100) |
| 185 | 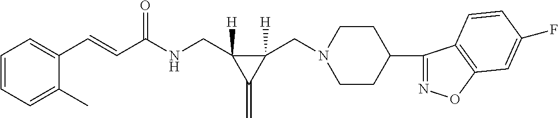 | 459.57 | 460 (100) |
| 186 | 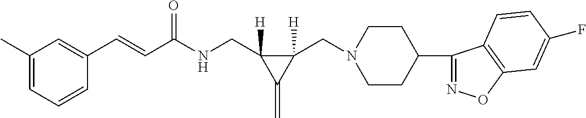 | 459.57 | 460 (100) |
| 187 | 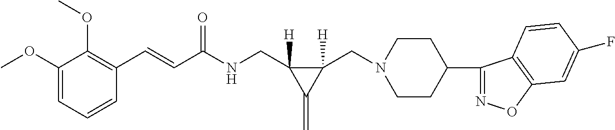 | 505.59 | 506 (100) |
| 188 | 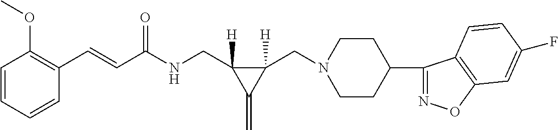 | 475.57 | 476 (100) |
| 189 | 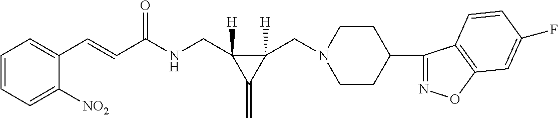 | 490.54 | 491 (100) |
| 190 | 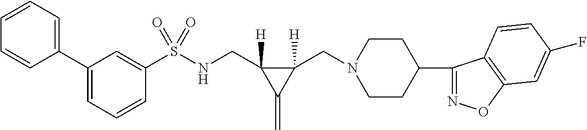 | 531.65 | 532 (100) |
| 191 | 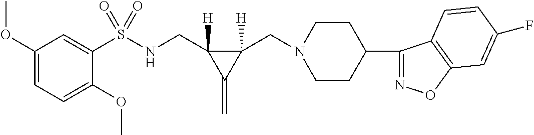 | 515.61 | 516 (100) |
| 192 | 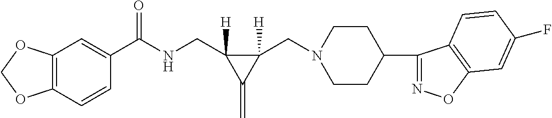 | 463.51 | 464 (100) |
| 193 | 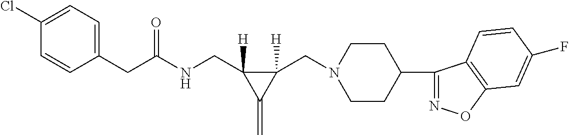 | 467.98 | 468 (100) |
| 194 | 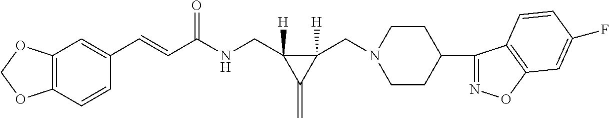 | 489.55 | 490 (100) |
| 195 | 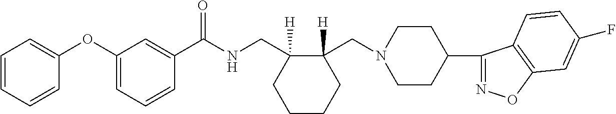 | 541.67 | 542 (100) |
| 196 | 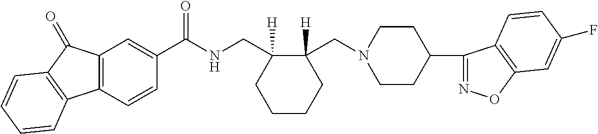 | 551.67 | 552 (100) |
| 197 | 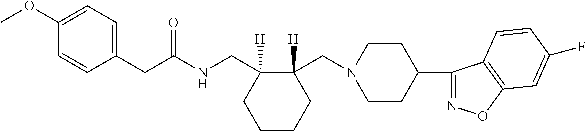 | 493.63 | 494 (100) |
| 198 | 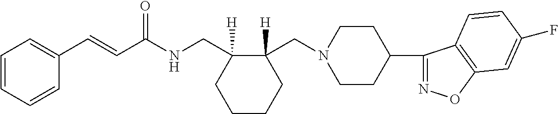 | 475.61 | 476 (100) |
| 199 | 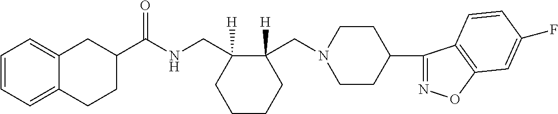 | 503.67 | 504 (100) |
| 200 | 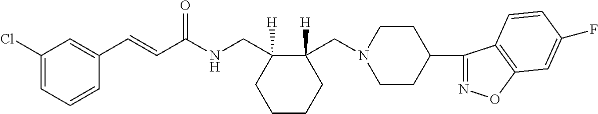 | 510.06 | 511 (100) |
| 201 | 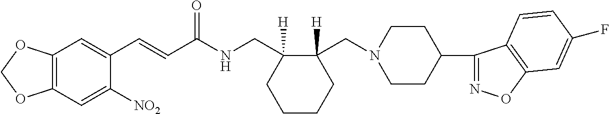 | 564.62 | 565 (100) |
| 202 | 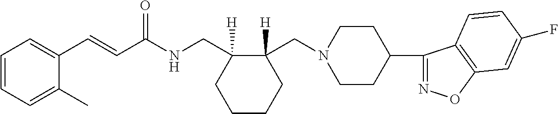 | 489.64 | 490 (100) |
| 203 | 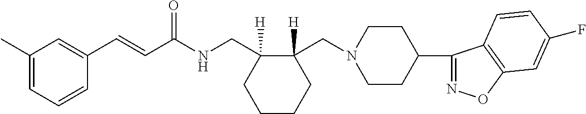 | 489.64 | 490 (100) |
| 204 | 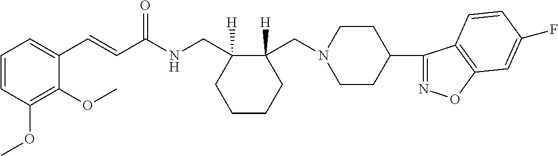 | 535.66 | 536 (100) |
| 205 | 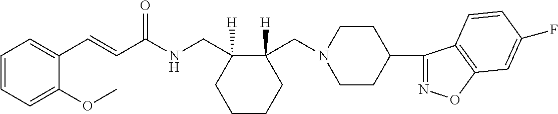 | 505.64 | 506 (100) |
| 206 | 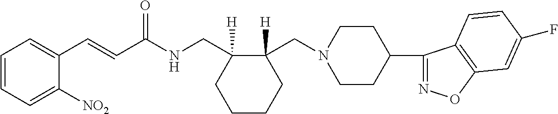 | 520.61 | 521 (100) |
| 207 | 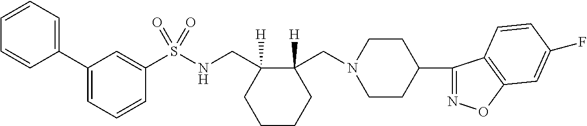 | 561.72 | 562 (100) |
| 208 | 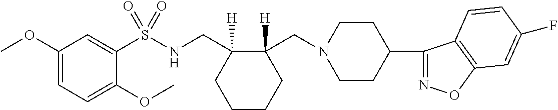 | 545.68 | 546 (100) |
| 209 | 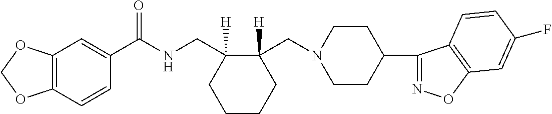 | 493.58 | 494 (100) |
| 210 | 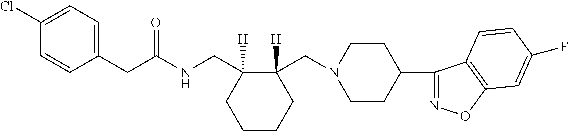 | 498.05 | 499 (100) |
| 211 | 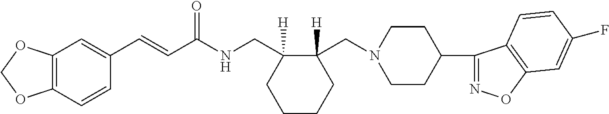 | 519.62 | 520 (100) |
| 212 | 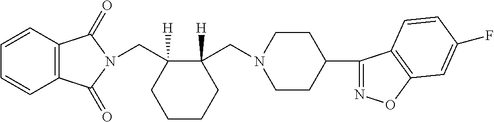 | 475.57 | 476 (100) |
| 213 | 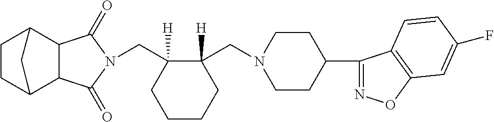 | 493.63 | 494 (100) |
| 214 | 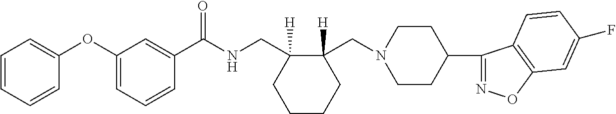 | 541.67 | 532 (100) |
| 215 | 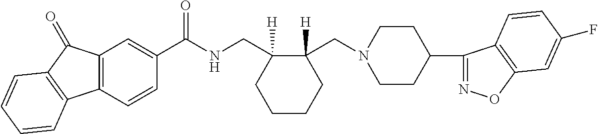 | 551.67 | 552 (100) |
| 216 | 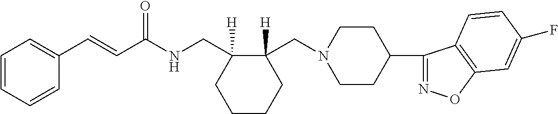 | 475.61 | 476 (100) |
| 217 | 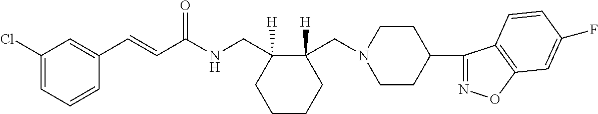 | 510.06 | 511 (100) |
| 218 | 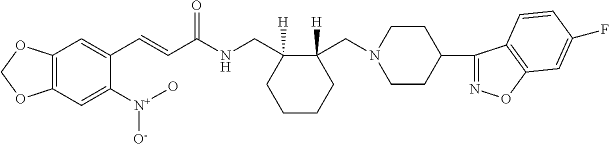 | 564.62 | 565 (100) |
| 219 | 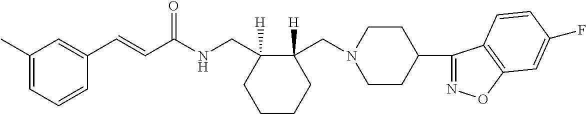 | 489.64 | 490 (100) |
| 220 | 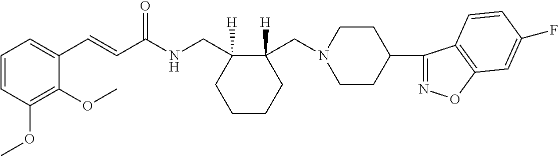 | 535.66 | 536 (100) |
| 221 | 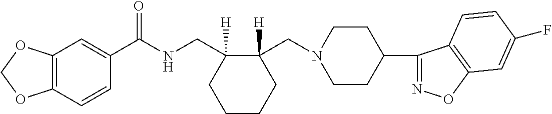 | 493.58 | 494 (100) |
| 222 | 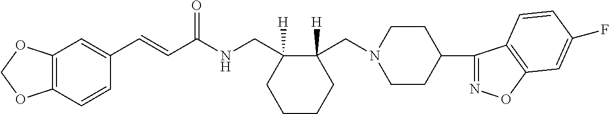 | 519.62 | 520 (100) |
| 223 | 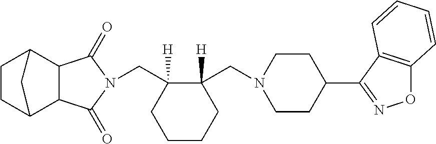 | 475.64 | 476 (100) |
| 224 | 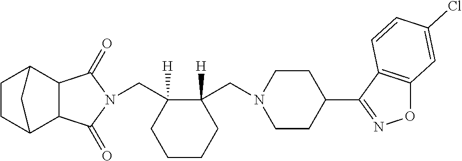 | 510.08 | 511 (100) |
| 225 | 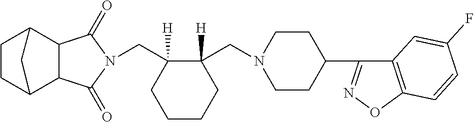 | 493.63 | 494 (100) |
| 226 | 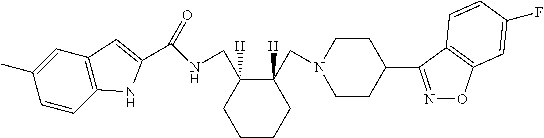 | 502.64 | 503 (100) |
| 227 | 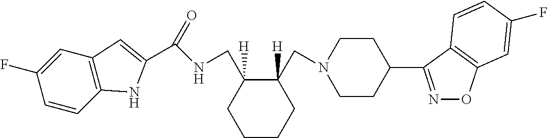 | 506.60 | 507 (100) |
| 228 | 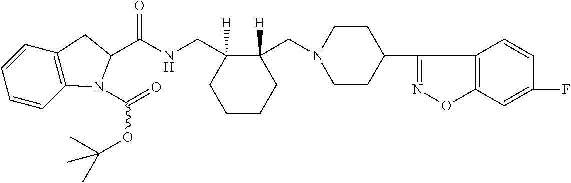 | 590.74 | 591 (100) |
| 229 | 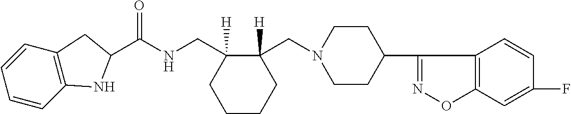 | 490.63 | 491 (100) |
| 230 | 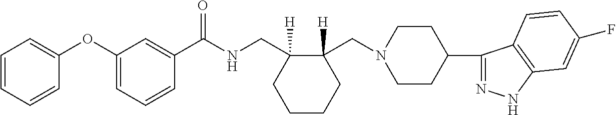 | 540.69 | 541 (100) |
| 231 | 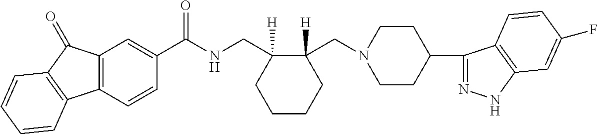 | 550.68 | 551 (100) |
| 232 | 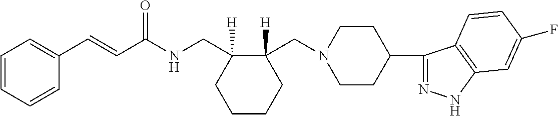 | 474.63 | 475 (100) |
| 233 | 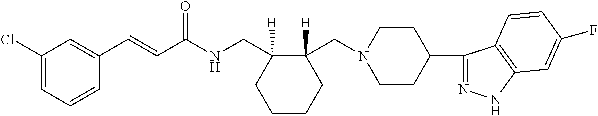 | 509.07 | 510 (100) |
| 234 | 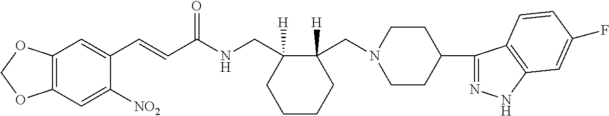 | 563.63 | 564 (100) |
| 235 | 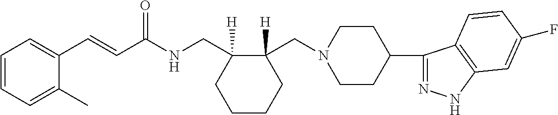 | 488.65 | 489 (100) |
| 236 | 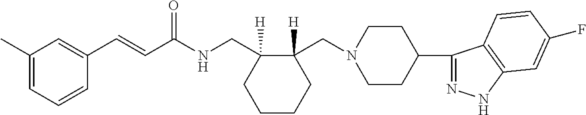 | 488.65 | 489 (100) |
| 237 | 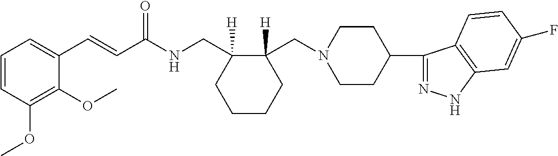 | 534.68 | 535 (100) |
| 238 | 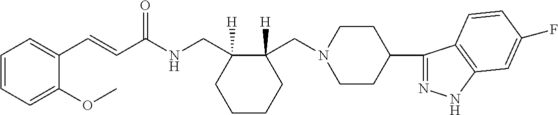 | 504.65 | 505 (100) |
| 239 | 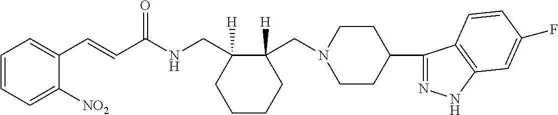 | 519.62 | 520 (100) |
| 240 | 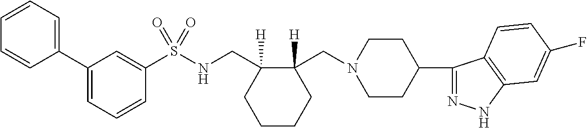 | 560.74 | 561 (100) |
| 241 | 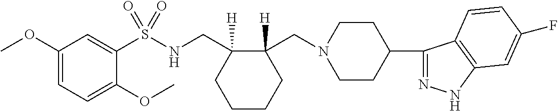 | 544.69 | 545 (100) |
| 242 | 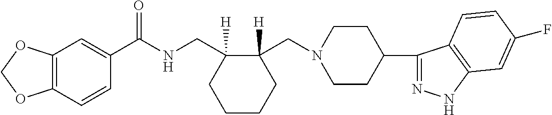 | 492.60 | 493 (100) |
| 243 | 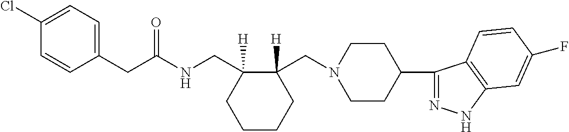 | 497.06 | 498 (100) |

### Examples 244-343

The following compounds can be prepared in a similar manner.

| Example No. | Structure |
|---|---|
| 244 | 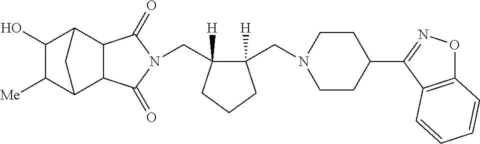 |
| 245 | 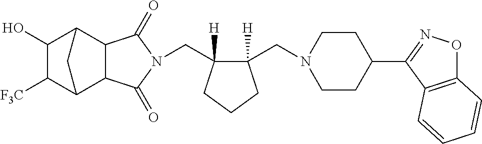 |
| 246 | 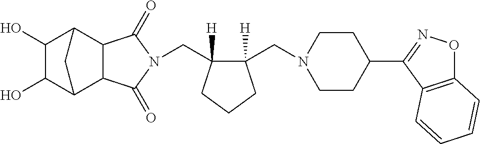 |
| 247 | 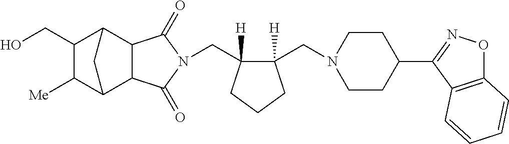 |
| 248 | 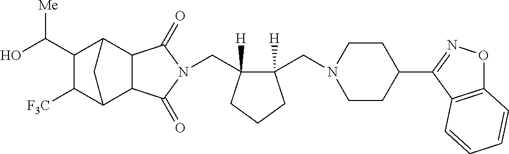 |
| 249 | 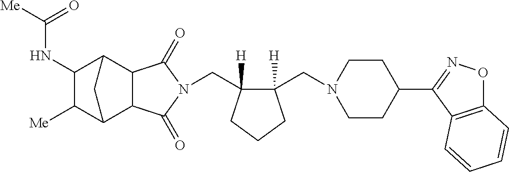 |
| 250 | 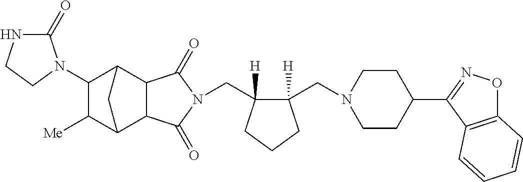 |
| 251 | 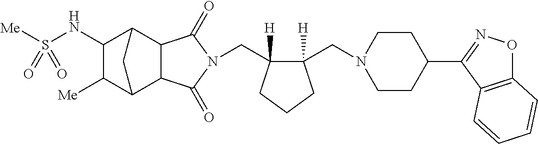 |
| 252 | 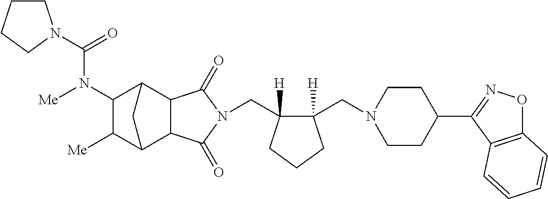 |
| 253 | 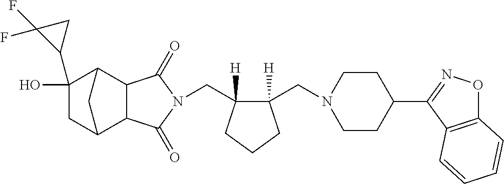 |
| 254 | 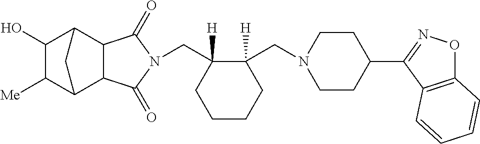 |
| 255 | 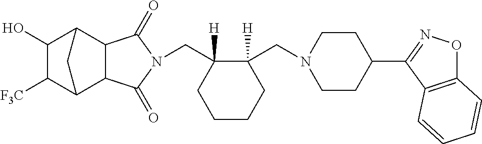 |
| 256 | 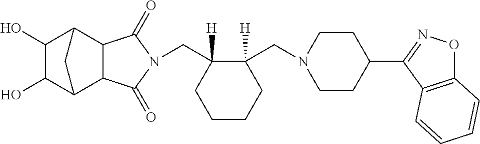 |
| 257 | 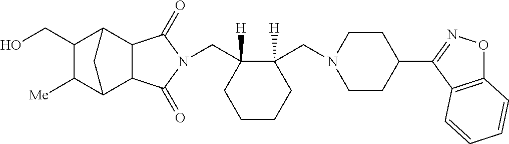 |
| 258 | 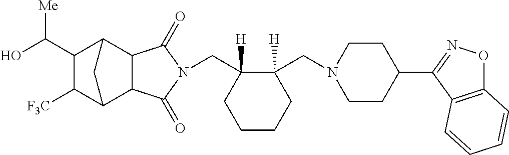 |
| 259 | 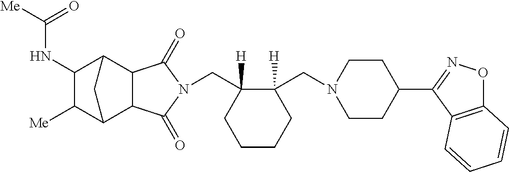 |
| 260 | 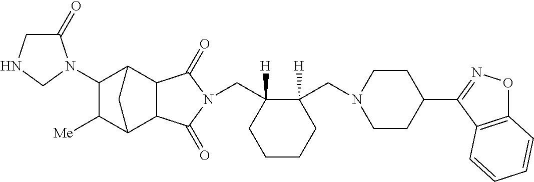 |
| 261 | 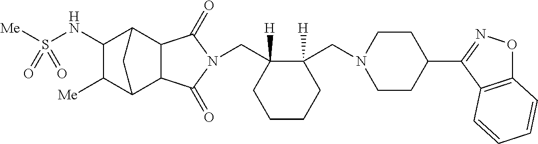 |
| 262 | 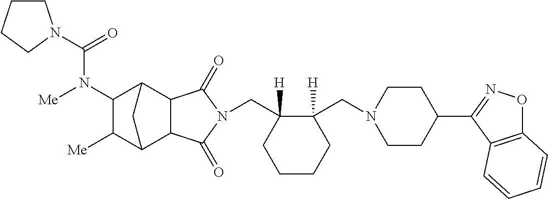 |
| 263 | 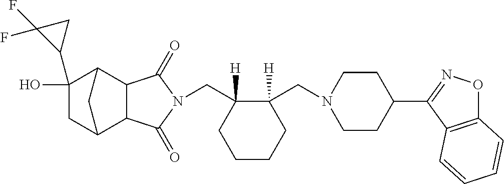 |
| 264 | 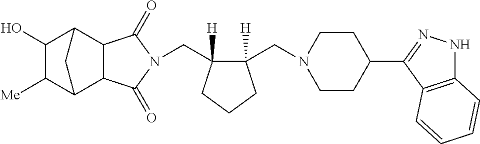 |
| 265 | 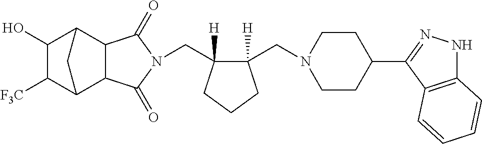 |
| 266 | 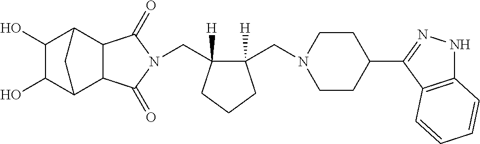 |
| 267 | 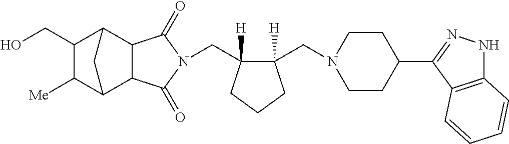 |
| 268 | 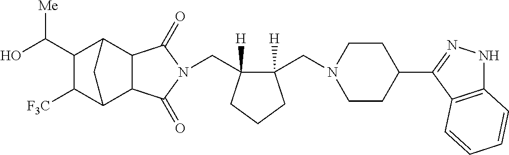 |
| 269 | 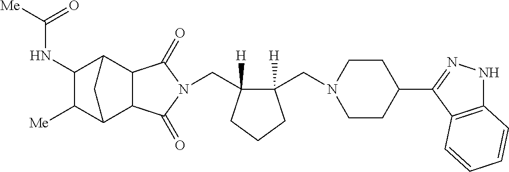 |
| 270 | 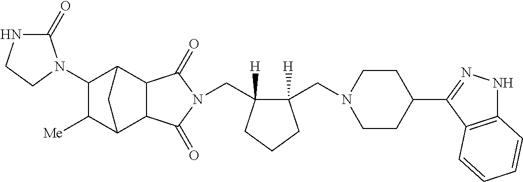 |
| 271 | 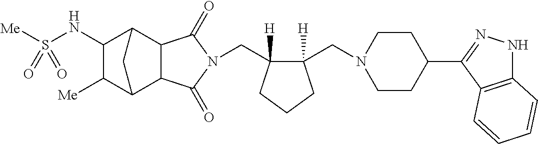 |
| 272 | 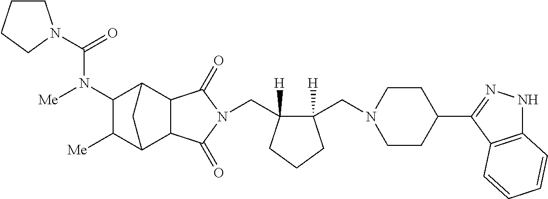 |
| 273 | 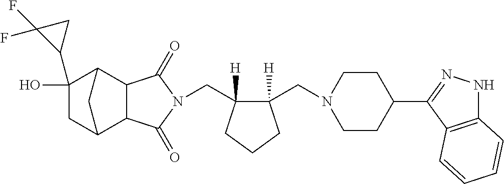 |
| 274 | 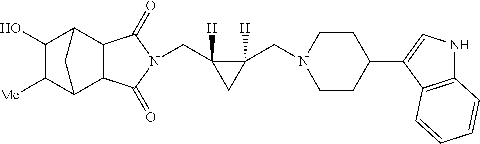 |
| 275 | 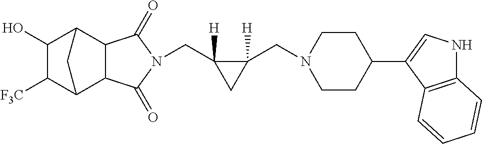 |
| 276 | 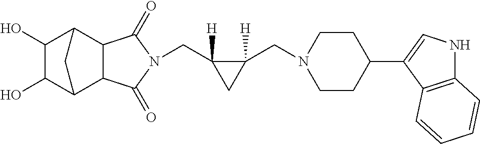 |
| 277 | 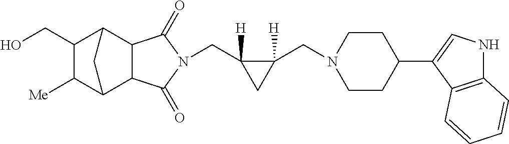 |
| 278 | 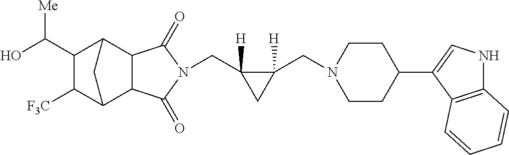 |
| 279 | 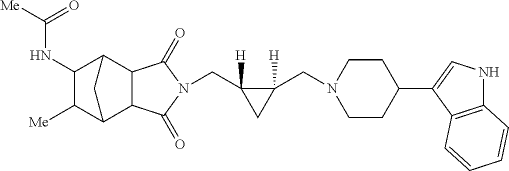 |
| 280 | 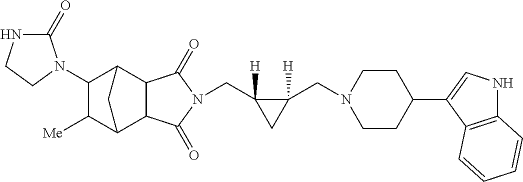 |
| 281 | 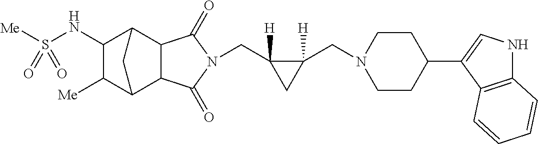 |
| 282 | 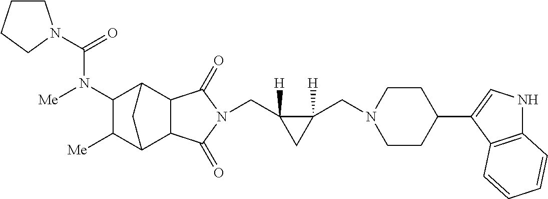 |
| 283 | 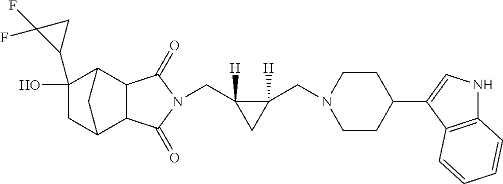 |
| 284 | 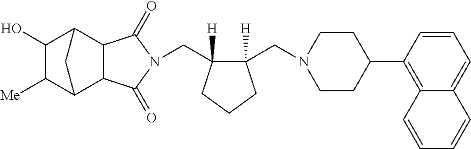 |
| 285 | 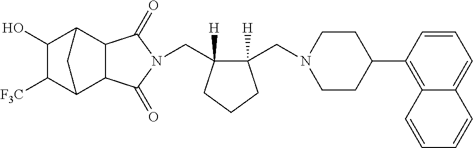 |
| 286 | 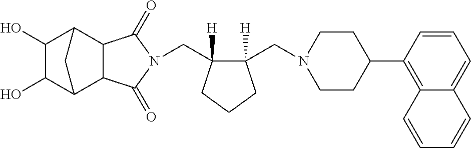 |
| 287 | 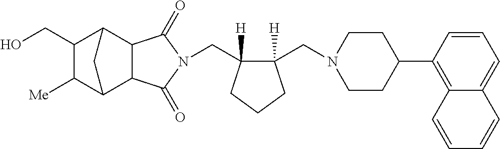 |
| 288 | 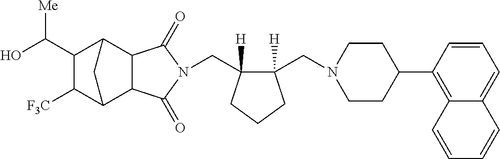 |
| 289 | 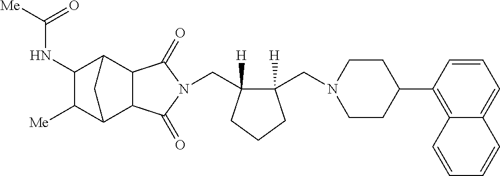 |
| 290 | 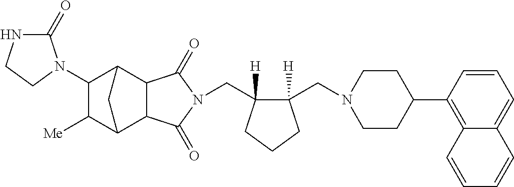 |
| 291 | 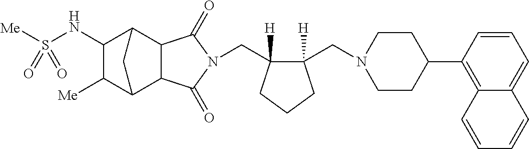 |
| 292 | 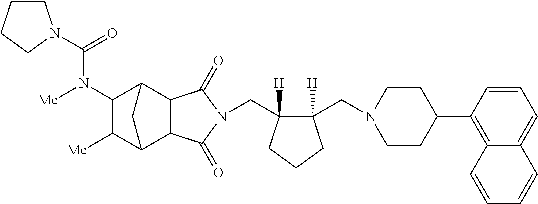 |
| 293 | 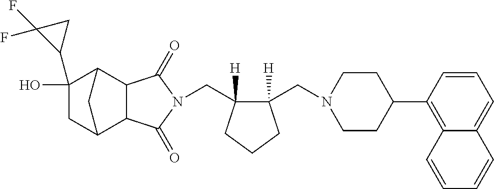 |
| 294 | 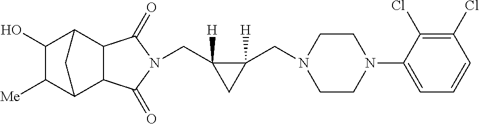 |
| 295 | 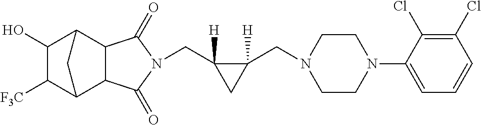 |
| 296 | 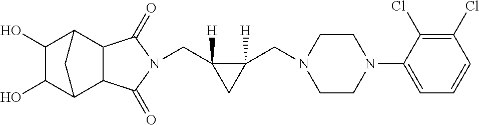 |
| 297 | 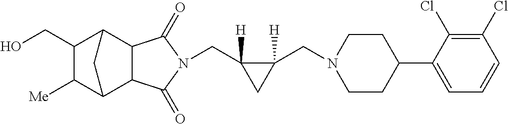 |
| 298 | 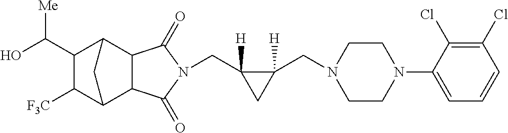 |
| 299 | 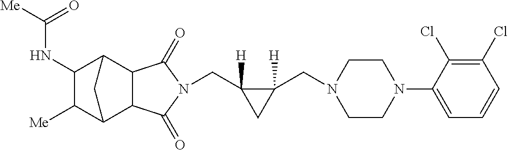 |
| 300 | 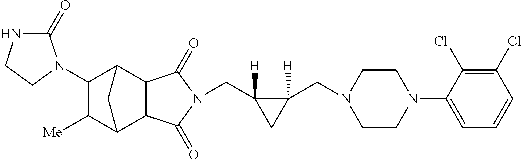 |
| 301 | 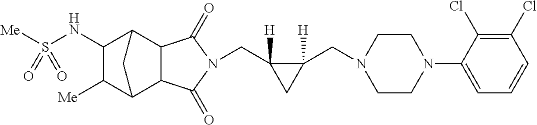 |
| 302 | 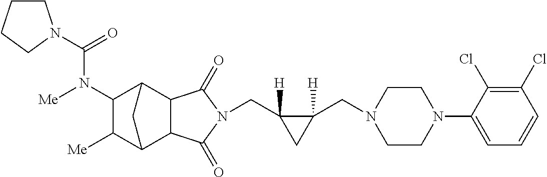 |
| 303 | 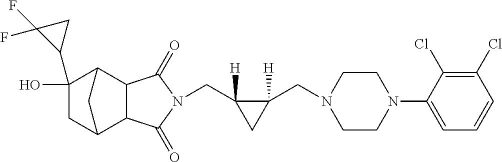 |
| 304 | 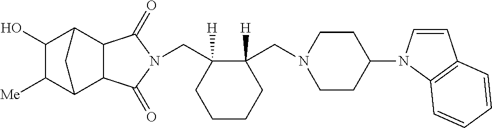 |
| 305 | 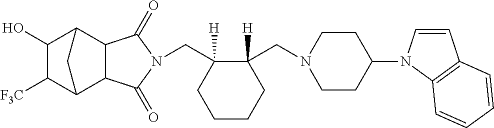 |
| 306 | 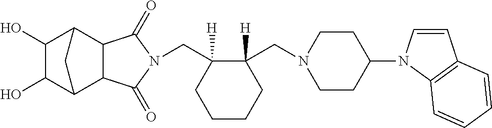 |
| 307 | 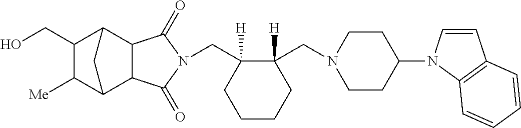 |
| 308 | 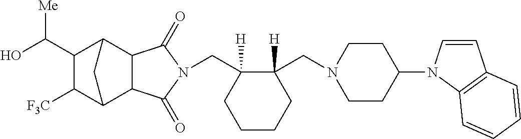 |
| 309 | 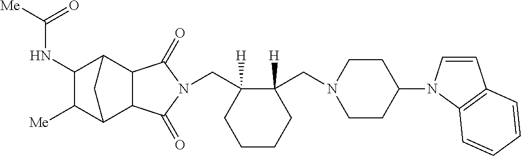 |
| 310 | 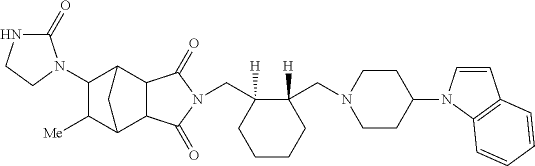 |
| 311 | 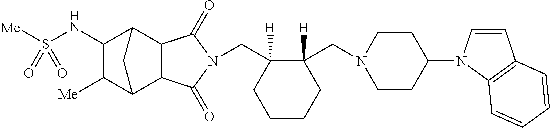 |
| 312 | 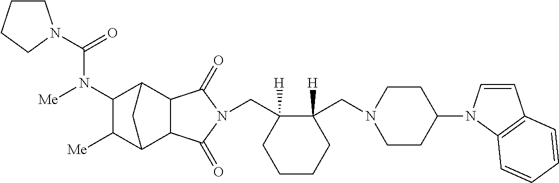 |
| 313 | 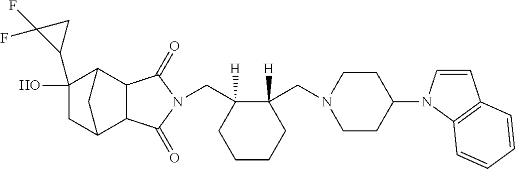 |
| 314 | 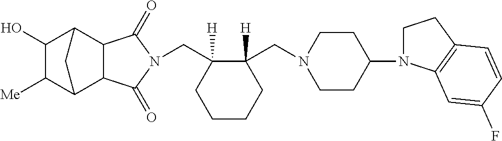 |
| 315 | 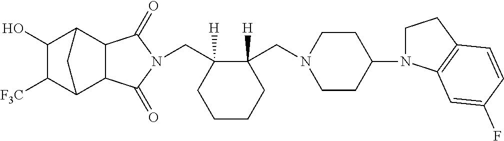 |
| 316 | 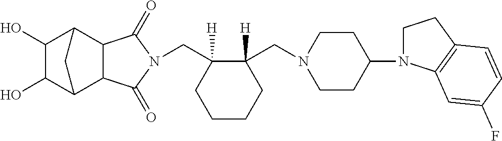 |
| 317 | 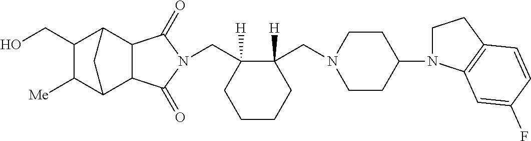 |
| 318 | 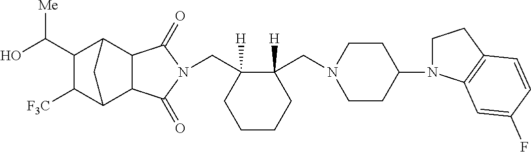 |
| 319 | 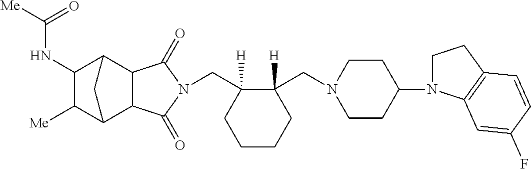 |
| 320 | 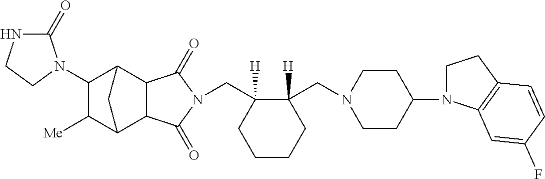 |
| 321 | 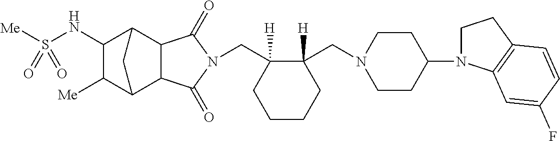 |
| 322 | 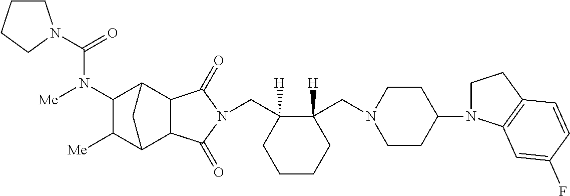 |
| 323 | 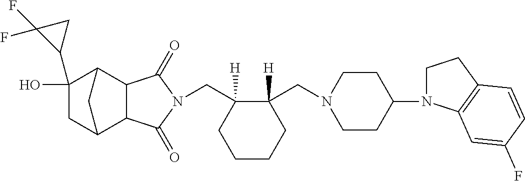 |
| 324 | 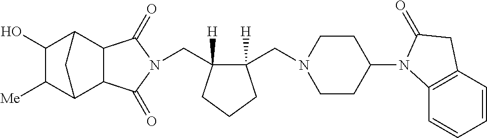 |
| 325 | 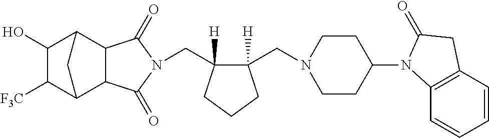 |
| 326 | 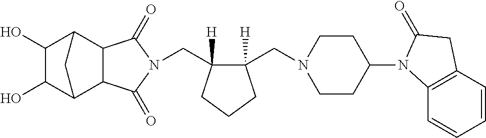 |
| 327 | 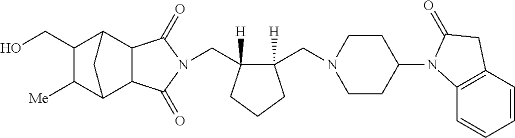 |
| 328 | 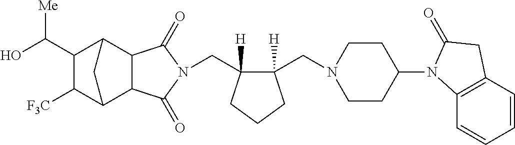 |
| 329 | 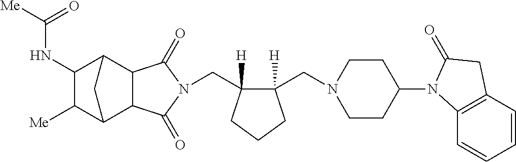 |
| 330 | 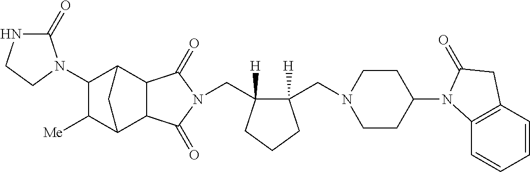 |
| 331 | 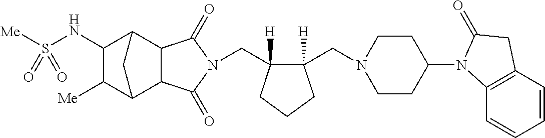 |
| 332 | 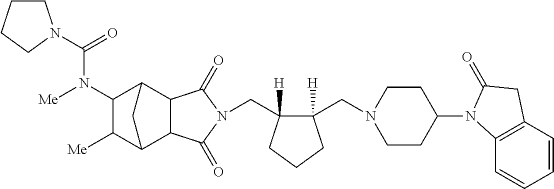 |
| 333 | 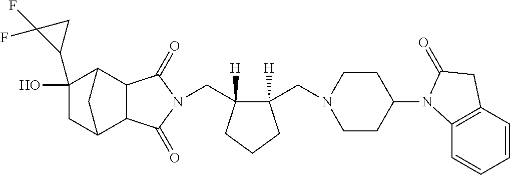 |
| 334 | 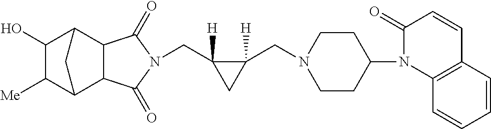 |
| 335 | 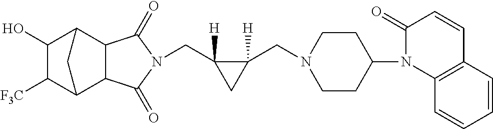 |
| 336 | 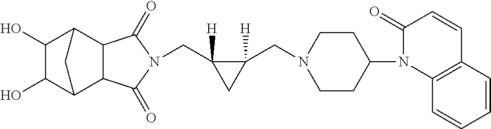 |
| 337 | 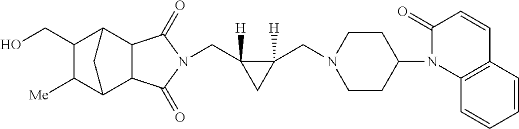 |
| 338 | 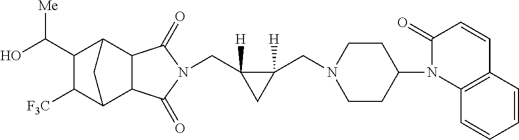 |
| 339 | 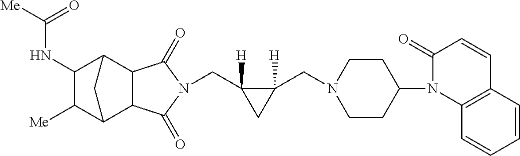 |
| 340 | 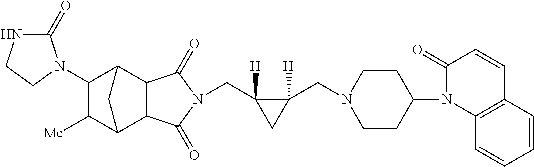 |
| 341 | 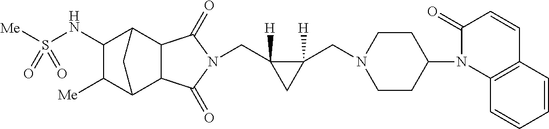 |
| 342 | 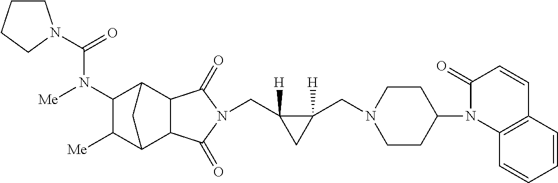 |
| 343 | 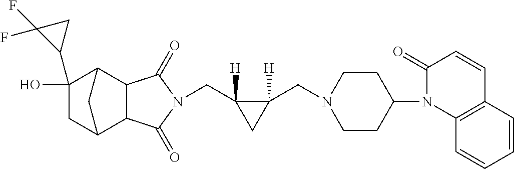 |

### Experiment 1

### Method

### (1) Antipsychotic Action (Anti D2 Activity) (In Vitro)

In general, there is clinically some correlation between antipsychotic action and antagonistic action for dopamine D2 receptor. The experimental method used herein is a binding assay for D2 receptor which is one of the tests evaluating D2 receptor action in vitro, According to the known method (e.g. Japan, J. Pharmacol., 53, 321-329 (1990)), the above-captioned experiment was carried out using [3H] spiperone, i.e., the binding amount of [3H] spiperone to the preparation cell membrane expressing human D2 receptor was measured, and then the binding inhibitory rate by the test compound (100 nM) was measured/calculated. The results are shown in the following table.

| | Binding inhibitory |
|---|---|
| Example | rate (%) for D2 |
| No. | receptor (100 nM) |
| 13 | 92 |
| 14 | 92 |
| 15 | 93 |
| 16 | 94 |
| 21 | 91 |
| 32 | 91 |
| 33 | 90 |
| 36 | 92 |
| 37 | 90 |
| 44 | 91 |
| 60 | 91 |
| 62 | 91 |
| 64 | 92 |
| 67 | 90 |
| 69 | 91 |
| 82 | 94 |
| 83 | 94 |
| 84 | 95 |
| 85 | 91 |
| 86 | 94 |
| 87 | 93 |
| 100 | 91 |
| 101 | 94 |
| 102 | 95 |
| 104 | 92 |
| 137 | 95 |
| 139 | 91 |
| 185 | 92 |
| 190 | 92 |
| 192 | 90 |
| 200 | 92 |
| 221 | 93 |
| 222 | 93 |
| 223 | 94 |
| 225 | 94 |
| 239 | 95 |
| 240 | 85 |
| 241 | 95 |
| 242 | 95 |
| 243 | 94 |

### (2) Antipsychotic Action (In Vivo)

The anti-methamphetamine test which is a typical in vivo test for evaluating antipsychotic action in the clinical study was carried out as follows. The test substance was intraperitoneally administered to a male rat, and 30 minutes later methamphetamine (1 mg/kg) was intraperitoneally administered to the male rat. Ten minutes after completing the administrations, the movement of the rat was measured with Supermex for 90 minutes, and the dose for 50% inhibition: ED₅₀ value was calculated. The result was shown in the table below.

| Example No. | Antidopaminergic action ED₅₀ (mg/kg) |
|---|---|
| 87 | 4.5 |
| 200 | 0.24 |

### (3) Side Effects

The cataleptic action which is a typical central nervous system side-effect of antipsychotic agents in the clinical study was evaluated as follows. The test substance was intraperitoneally administered to a male rat. One hour later, the rat was made to hold on a pole three times which is horizontally set at a height of 9 cm. When the rat retained the unnatural stretched pose for 30 seconds even one time out of three times, the rat was evaluated as cataleptic positive. The dose for inducing 50% the rats to the catalepsy: ED₅₀ value was calculated. The result was shown in the table below.

| Example No. | Cataleptic ED₅₀ (mg/kg) |
|---|---|
| 87 | >135 |
| 200 | >7.2 |

### EFFECT OF THE INVENTION

The compounds of the present invention and acid additive salts thereof exhibited a potent psychotropic action, Especially, it has become clear that the compounds of the present invention and acid additive salts thereof exhibit an excellent effect for improving a broad spectrum of schizophrenia such as positive symptom, negative symptom, and cognitive dysfunction, while never almost causing abnormal electrocardiogram, weight gain, etc., thus are very safe medicaments.

What is claimed is:

1. A cycloalkane derivative of formula [1]: 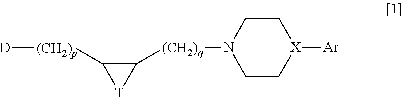
wherein
p and q are independently 1 or 2;
T is —(CH₂)ₙ— wherein n is 1 to 4, or —C(═CH₂)—;
D is the group of formula [2]: 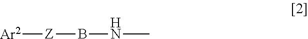
wherein Ar² is an aromatic heterocyclyl group or an aromatic carbocyclyl group wherein the aromatic heterocyclyl group and the aromatic carbocyclyl group may be substituted with 1 or 2 substituents selected independently from the group consisting of nitro group, cyano group, halogen atom, lower alkyl group, lower alkoxy group, trifluoromethyl group, trifluoromethoxy group and phenoxy group, and further the lower alkyl group, the alkoxy group and the phenoxy group may be substituted with one or more halogen atoms which are the same or different;
B is carbonyl group or sulfonyl group,
Z is single bond, lower alkylene, lower alkenylene, or ethynylene,
the group of formula [3]: 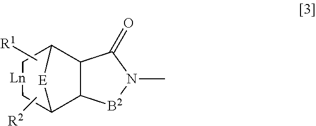
wherein B² is carbonyl group or sulfonyl group;
Ln is single or double bond;
E is lower alkylene which may be optionally substituted with one or two lower alkyl groups which are the same or different, oxygen atom, or two hydrogen atoms which are attached at the both ends (i.e., E is not a bridge);
R¹ and R² are independently hydrogen atom, hydroxy, lower alkyl group, or lower cycloalkyl group, which may be independently connected to any one of the carbon atoms which compose the ring of formula [3], or R¹ and R² may be connected to the same carbon atom if possible, wherein the lower alkyl group and the lower cycloalkyl group may be substituted with one or more substituents selected independently from the group consisting of hydroxy group and fluorine atom, or
the group of formula [4]: 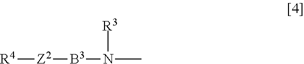
wherein B³ is carbonyl group or sulfonyl group;
Z² is single bond, oxygen atom, or —NR⁵—;
R³, R⁴ and R⁵ are independently hydrogen atom or lower alkyl, or R³ and R⁴, or R⁴ and R⁵ may be connected together directly or via lower alkylene to form a ring;
X is N, CH or C(OH); and
Ar is aromatic heterocyclyl group, aromatic hydrocarbon group, benzoyl, or phenoxy, wherein the aromatic heterocyclyl group, the aromatic hydrocarbon group, the benzoyl, and the phenoxy may be substituted with one or more substituents selected independently from the group consisting of lower alkyl, lower alkoxy and halogen atom;
provided that Ar is not benzisothiazolyl group when D is the substituent of formula [3],
or an acid additive salt thereof.

2. The cycloalkane derivative of claim 1
wherein
p and q are 1,
T is —(CH₂)ₙ— wherein n is 1 to 4,
D is the group of formula [2]: 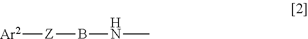
wherein Ar² and B are defined as claim 1, and Z is single bond, methylene, vinylene or ethynylene, or
the group of formula [3]: 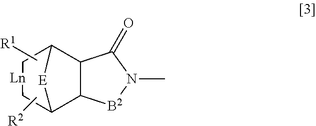
wherein B², E, R¹, R² and Ln are defined as claim 1, and
X is N or CH,
or an acid additive salt thereof.

3. The cycloalkane derivative of claim 2
wherein
T is —(CH₂)ₙ— wherein n is 3 or 4,
D is the group of formula [2]: 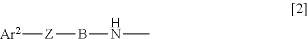
wherein Ar² and B are defined as claim 2, and Z is single bond, methylene or vinylene,
or an acid additive salt thereof.

4. The cycloalkane derivative of claim 2 or 3 wherein Z is single bond or vinylene, or an acid additive salt thereof.
